SEQUENCE LISTING (11 sequences)

SEQ ID NO: 1 - protein, 30 aa, Artificial Sequence
  Description of Artificial Sequence Synthetic Human immunodeficiency virus and Homo sapiens construct
RKKRRQRRRPPQCAAVALLAPAVLLALLAP

SEQ ID NO: 2 - protein, 16 aa, Xenopus laevae
RQIKIWFQNRRMKWKK

SEQ ID NO: 3 - protein, 27 aa, Artificial Sequence
  Description of Artificial Sequence Synthetic galanin (Homo sapiens) and mastoparan (derived from wasps) construct
GWTLNSAGYLLGKINLKALAALAKKIL

SEQ ID NO: 4 - protein, 18 aa, Artificial Sequence
  Description of Artificial Sequence Synthetic Peptide
KLALKLALKALKAALKLA

SEQ ID NO: 5 - protein, 16 aa, Artificial Sequence
  Description of Artificial Sequence Synthetic Peptide
KLWSAWPSLWSSLWKP

SEQ ID NO: 6 - protein, 28 aa, Artificial Sequence
  Description of Artificial Sequence Synthetic Human immunodeficiency virus and Homo sapiens construct
AAVALLPAVLLALLAPRKKRRQRRRPPQ

SEQ ID NO: 7 - protein, 33 aa, Artificial Sequence
  Description of Artificial Sequence Synthetic Peptide
LLETLLKPFQCRICMRNFSTRQARRNHRRRHRR

SEQ ID NO: 8 - protein, 29 aa, Unknown
  Description of Unknown Organism TAT peptide attached to fusion domain, both of viral origins
RRRQRRKRGGDIMGEWGNEIFGAIAGFLG

SEQ ID NO: 9 - protein, 28 aa, Artificial Sequence
  Description of Artificial Sequence Synthetic Peptide
KETWWETWWTEWSQPGRKKRRQRRRPPQ

SEQ ID NO: 10 - protein, 22 aa, Artificial Sequence
  Description of Artificial Sequence Synthetic Peptide
GLGSLLKKAGKKLKQPKSKRKV

SEQ ID NO: 11 - protein, 36 aa, Homo sapiens
KGSKKAVTKAQKKDGKKRKRSRKESYSVYVYKVLKQ

(19) United States
(12) Patent Application Publication
(10) Pub. No.: US 2006/0003989 A1
(43) Pub. Date: Jan. 5, 2006

(54) Compositions and methods using acetylcholinesterase (ACE) inhibitors to treat central nervous system (CNS) disorders in mammals

(75) Inventors: Steven C. Quay, Edmonds, WA (US); Henry R. Costantino, Woodinville, WA (US); Michael E. Houston JR., Sammamish, WA (US); Alexis Kays Leonard, Maple Valley, WA (US)

Correspondence Address: NASTECH PHARMACEUTICAL COMPANY INC, 3450 MONTE VILLA PARKWAY, BOTHELL, WA, 98021-8906, US

(73) Assignee: Nastech Pharmaceutical Company Inc.

(21) Appl. No.: 11/112,950

(22) Filed: Apr. 22, 2005

Related U.S. Application Data
(63) Continuation-in-part of application No. 10/831,031, filed on Apr. 23, 2004.
Application No. 10/831,031 is a continuation-in-part of application No. 10/439,108, filed on May 15, 2003.
(60) Provisional application No. 60/382,122, filed on May 21, 2002.

(51) Int. Cl. A61K 31/55

(52) U.S. Cl. 514/214.03

(57) ABSTRACT

Methods and compositions of the invention employ acetylcholinesterase (ACE) inhibitors to prevent and treat diseases and other disorders of the central nervous system (CNS), including Alzheimer's disease. ACE inhibitors are administered for targeted delivery to the CNS, for example by intranasal delivery. The methods and compositions of the present invention yield therapeutic concentrations of ACE inhibitors in a CNS tissue or compartment without the attendant disadvantages, risks and side effects of oral or injection delivery. Exemplary ACE inhibitors for use within the invention include galantamine and various salts and derivatives of galantamine. Carboxylate salts of galantamine (e.g., galantamine gluconate, galantamine lactate, galantamine citrate and galantamine glucarate) described herein exhibit a significant increase in solubility compared to other forms of galantamine, such as galantamine hydrobromide.

### CROSS REFERENCES TO RELATED APPLICATIONS

This is a continuation-in-part of, and claims priority under Title 35, U.S. Code, §120 to, co-pending U.S. patent application Ser. No. 10/831,031 filed on Apr. 23, 2004, which is a continuation-in-part of U.S. patent application Ser. No. 10/439,108 filed on May 15, 2003, which claims the benefit under 35 U.S.C § 119(e) of U.S. Provisional Application No. 60/382,122 filed on May 21, 2002. Each of the foregoing priority disclosures are incorporated herein by reference.

### BACKGROUND OF THE INVENTION

Acetylcholinesterase (ACE) inhibitors comprise an important class of drugs for the prevention and treatment of diseases and disorders of the central nervous system (CNS). Diseases amenable for treatment employing ACE inhibitors include, inter alia, neurological conditions associated with memory loss, cognitive impairment and dementia in mammals, including Alzheimer's Disease, Parkinson's-type dementia, Huntington's-type dementia, Pick's-type dementia, CJ-type dementia, AIDS-related dementia, Lewy Body dementia, Rett's syndrome, epilepsy, brain malignancies or tumors, cognitive disorder associated with multiple sclerosis, Down's syndrome, progressive supranuclear palsy, certain forms of schizophrenia, depression, mania and related psychiatric conditions, Tourette's syndrome, mysasthenia gravis, attention deficit disorder, autism, dyslexia, forms of delirium, or dementia as a sequela to vascular stroke or cranial bleeding and brain injury, in their chronic, acute and relapsing forms.

Pathological changes in Alzheimer's disease involve degeneration of cholinergic neurons in the subcortical regions and of neuronal pathways that project from the basal forebrain, particularly Meynert's nucleus basalis to the cerebral cortex and hippocampus (Robert P H et al. 1999. “Cholinergic Hypothesis and Alzheimer's Disease: The Place of Donepezil (Aricept),” Encephale 5:23-5 and 28-9). These pathways are thought to be intricately involved in memory, attention, learning, and other cognitive processes.

The earliest signs of dementia appear as mild cognitive and memory impairment. This occurs progressively in underlying conditions such as Alzheimer's disease and suddenly in dementia related to vascular embolism or bleeding aneurism. Dementia in its advanced form is associated with aggressive behavior, irrational and paranoid ideation, loss of memory, loss of sense of smell, and often with cataracts. Non-vascular dementia is always related to abnormal deposition of a particular protein in the nerve bodies and sheaths. In Alzheimer's the abnormal deposits of amyloid protein are called plaques. Plaques containing other unique proteins appear in Parkinson's disease, Huntington's disease, Pick's disease and in prion disease associated with cognitive impairment. These plaques can be identified histologically at autopsy. Dense tangles of the tau protein are also observed intracellularly in dementia. Certain alleles at several loci, most notably the ApoE e4 allele, have been noted to have a higher incidence of late-onset dementia. Late and early onset forms of the disease are differentiated by the age at onset; 65 years of age can be taken as a cutoff for “early” onset versus late onset disease.

Vascular dementia may present with unique symptoms, such as gait abnormality and urinary incontinence, generally due to multiple cerebral infarctions, intracerebral hemorrhage, strokes, or infectious vasculitis of Lyme's disease, and autoimmune vasculitis of lupus erythrematosis, among other related conditions.

Of all testing methods for dementia and delerium, the most objective early measure is cognitive testing. Standardized testing in humans may be performed using the Reye Auditory Verbal Learning Test, the Mini-Mental State Exam (MMSE) the Weschler Logical Memory Test, or the Selective Reminding Test, among others. The cognitive subscale is also a major indication in the Alzheimer's Disease Assessment Scale (ADAS-cog), and simultaneously assesses short term memory, orientation in place and time, attention span, verbal ability and praxis. ADAS-cog testing is used diagnostically, higher scores indicating cognitive impairment, but may also be used to evaluate success in treatment. Reduced scores following treatment with tacrine, donepezil and the longer-acting rivastigmine have been noted.

It is believed that ACE inhibitors exert their therapeutic effect in the CNS by enhancing cholinergic function, i.e., by increasing the concentration of acetylcholine through reversible inhibition of its enzymatic hydrolysis by the cholinesterases. This pharmacotherapeutic approach also has some value in treatment of nicotine withdrawal and sleep apnea, as well as the dementia and delirium states described above.

The three ACE inhibitor drugs presently on the market are delivered orally in the form of tablets and capsules. During oral delivery, drug passes down the digestive tract and is absorbed into blood capillaries of the duodenum and ileum, enters the portal vein, and is then transported to the liver before reaching the target organ, the brain. Unfortunately, oral delivery of acetylcholinesterase inhibitors is associated with several disadvantages, including, inter alia:
(1) hepatic first pass metabolism and clearance,
(2) gastrointestinal destruction of the drug by digestive enzymes and by the acidic pH conditions of the digestive tract;
(3) inferior and unpredictable uptake and bioavailability, especially as affected by food ingestion; and
(4) serious adverse effects, including nausea, vomiting, loose stools, diarrhea, anorexia and in severe cases, irreparable esophageal tears.

The possibility of injection or topical application of a cholinesterase has been reported, and an injectable dosage form of donepezil is described in PCT/US01/07027. However, injectables are not suitable for many patients with low muscle mass, are inherently dangerous in patients who lack a fully functional immune system, and require extra expense, time and training. When one considers that a dose of these drugs may be required up to 4 times a day, the invasive route of administration by injection is undesirable unless the patient is hospitalized and has a central IV line open at all times.

Other options for delivery of ACE inhibitors include inhalation and absorption via the pulmonary mucosa. Related delivery methods are reported in PCT/US01/07027 (“Novel Methods Using Cholinesterase Inhibitors”). This report discloses that aerosol sprays and fine powdered solid dosage forms can reach the lung when administered by pressurized spray or ventilatory support through the nose or mouth. These inhalation dosage forms are formulated with propellants for use in insufflators or nebulizers. However, in order to reach the large surface area of the alveoli, special equipment is often needed. The formulations require propellants or other means of achieving a very fine mist or powder with particle size less than 10 μm diameter. The mist or powder is then administered to the lungs via the mouth or the nose using intubation. If possible, the patient must be trained to actively inhale during dosing, or pressurized respiratory assist may be required, as in patients suffering from asthma, chronic obstructive pulmonary disease, emphysema or physical debilitation due to ageing. Generally, in these patients, the assistance of a trained technician is required to achieve efficacious dosing. Unfortunately, consistency of dosing with mists or powders has been problematic, and most research has been directed to high-density powders because liquid solutions of drug, especially aqueous solutions, do not have the low surface tension needed to form a dense and slow settling microaerosol suitable for inhalation therapy. The methods employed do not readily lend themselves to home dosing of elderly patients and are inherently expensive due to the costs associated with the specialized delivery devices and route of administration. Metered dose inhalation is one of the most complex drug delivery systems on the market. More information about pressurized devices used for aerosol inhalation drug delivery is provided in Remington: The Science and Practice of pharmacy, 19^{th} ed. Chapter 95 “Aerosols”, and a descriptive definition of the inhalation route for drug delivery is provided in Chapter 41, “Drug Absorption, Action and Disposition” under the heading of Absorption of Drugs: Inhalation Route.

There have been various attempts to deliver drugs intranasally, i.e. without formulation or devices for inhalation, in the treatment of brain disorders. For example, U.S. Pat. No. 6,180,603 discloses a method for actively and transaxonally delivering therapeutic agents that are autologous counterparts of endogenous proteins, peptides and complex lipids (all of which are native to the brain) via interneuronal transport in nerve cell bodies and membranes. This report discloses the transport of insulin, insulin-like growth factors, nerve growth factors, gangliosides, phosphatidylserine, brain-derived neurotrophic factors, fibroblast growth factors, glia-derived nexins, ciliary neurotrophic factors, and cholinergic enhancing factors, via the axon of the olfactory nerve. However, there is no disclosure or teaching of any useful intranasal delivery by this mechanism of non-naturally occurring, xenogenic drugs including synthetic heterocyclic amines, substituted piperidines, and substituted phenols, further encompassing acetylcholinesterase inhibitors, and, in particular, xenogenic, non-native acetylcholinesterase inhibitors. No disclosure is made of excipients useful for increasing paracellular and transcellular uptake of drugs, including ACE inhibitors.

In view of the foregoing, there remains an important need in the art for improved tools and methods to more effectively deliver ACE inhibitors to prevent and treat diseases and disorders of the CNS. Such tools and methods should avoid the toxicity and low bioavailability associated with oral delivery, and the expense, training and difficulty of dosing by injection and inhalation therapy.

### SUMMARY OF THE INVENTION

The present invention fulfills these needs and satisfies additional objects and advantages by providing novel pharmaceutical compositions and therapeutic methods employing one or more acetylcholinesterase (ACE) inhibitors to treat and/or prevent diseases or conditions of the central nervous system (CNS) in mammals. Exemplary pharmaceutical compositions of the invention for these purposes may comprise a liquid or gel solution or powder formulation for nasal administration comprising at least one ACE inhibitor and at least one permeation-enhancing agent to facilitate transmucosal drug uptake.

Diseases amenable for treatment using the methods and compositions of the invention, include, for example, Alzheimer's disease and other neurological conditions associated with cognitive impairment in mammalian subjects.

Within exemplary formulations and methods of the invention, an effective ACE inhibitor is selected from known ACE inhibitors or novel ACE inhibitor candidates—including but not limited to exemplary ACE inhibitors donepezil, tacrine, rivastigmine, galantamine, and pharmaceutically-acceptable salts and derivatives thereof. Typically, the ACE inhibitor is substantially free of native neurobiomolecules.

In other embodiments, methods for treating or preventing a CNS disease or condition in a mammal in need of treatment by therapeutic administration of an ACE inhibitor are provided, which generally involve administering a pharmaceutical composition containing a liquid or gel solution or powder formulation for nasal administration comprising at least one ACE inhibitor, and coordinately administering at least one permeation-enhancing agent that promotes transmucosal drug uptake. Within these methods, the ACE inhibitor(s) and permeation enhancing agent(s) may be administered simultaneously (e.g., in a single formulation), consecutively, or separately. In more detailed embodiments, the ACE inhibitor(s) and permeation enhancing agent(s) are administered intranasally.

In other detailed embodiments, the permeation-enhancing agent is a permeation-enhancing peptide agent that facilitates delivery of small molecule drugs, including small molecule ACE inhibitors, across mucosal epithelial barriers.

In other embodiments, the invention provides methods and compositions comprising novel salt forms of ACE inhibitors, exemplified by novel salt forms of galantamine. Within exemplary aspects, novel carboxylate salts of galantamine are provided which possess improved solubility characteristics in comparison to other forms of galantamine, rendering the formulations and methods useful for intransal delivery of galantamine to treat CNS disorders in mammals.

In other embodiments, the invention is directed to an administering device, preferably a nasal dispenser or pump for use with said composition, which may optionally be a multi-dose device. In further embodiments, the invention is directed to an article of manufacture comprising the pharmaceutical composition of the invention in a childproof package suitable for sale and distribution.

### BRIEF DESCRIPTION OF THE DRAWINGS

FIG. 1 shows a plot of donepezil cerebral spinal fluid (CSF)/plasma ratio in a rat, following single-dose nasal administration at time zero, followed by dual measurements of CSF and plasma at 5, 10, 30, and 60 minutes.

FIG. 2 shows the ultraviolet light (UV) absorbance of galantamine fractions described in Example 15.

FIG. 3 shows the effects of 25-100 μM PN159 on 40 mg/ml Galantamine lactate in vitro permeation of an epithelial monolayer.

FIG. 4 illustrates the effects of 25-100 μM PN159 with 40 mg/ml Galantamine lactate on in vitro TER.

FIG. 5 shows the effects of 25-100 μM PN159 with 40 mg/ml Galantamine lactate on in vitro cell viability, as measured by MTT.

FIG. 6 illustrates the effects of 25-100 μM PN159 with 40 mg/ml Galantamine lactate on in vitro cytoxicity, as measured by LDH.

FIG. 7 shows plasma galantamine levels after administration via different routes in canine subjects. Intranasal (diamonds); Oral Solution (triangles): Oral Tablet (squares).

FIG. 8 shows galantamine levels in CSF after administration via different routes in canine subjects. Oral Route (squares): Nasal Route (circles).

FIG. 9 shows plasma galantamine levels after administration via different routes in ferrets (P less than 0.0001).

FIG. 10 shows the incidence of retching after administration of galantamine via different routes in ferrets.

### DESCRIPTION OF EXEMPLARY EMBODIMENTS OF THE INVENTION

The present invention provides methods and compositions for improved delivery of acetylcholinesterase (ACE) inhibitors to the central nervous system (CNS). Within exemplary embodiments, the invention targets delivery of ACE inhibitors for prevention and treatment of CNS diseases and conditions in mammalian subjects via paracellular, intranasal delivery. The rich vascular plexus of the nasal cavity of mammals provides a direct route into the bloodstream for ACE inhibitors. Due to direct absorption into the bloodstream, problems of gastrointestinal destruction and hepatic first pass metabolism are avoided, thereby improving bioavailability of ACE inhibitor drugs relative to oral delivery. Thus, the methods and compositions of the invention provide higher bioavailability and maximum concentration in the CNS compared to other modes of delivery, without the same attendant disadvantages and side effects.

The pharmaceutical compositions of the invention containing ACE inhibitors and at least one permeation-enhancing agent can be delivered in effective quantities via intranasal administration methods as disclosed herein. Although these cationic drugs are not native to the body, and there is no prior disclosure of preferential delivery of ACE inhibitors to the brain or any organ (other than the liver for metabolism and excretion), the compositins and methods of the invention yield effective intranasal delivery of ACE inhibitors to the CNS. Within exemplary embodiments, the ACE inhibitors are delivered intranasally and their uptake is substantially paracellular. Because the enhancement of delivery according to thse methods and compositions is at least partially paracellular, the drug rapidly enters the cerebrospinal fluid (CSF) and blood for distribution throughout the brain.

The pharmaceutical compositions of the invention minimize the transport of the ACE inhibitors from the nasal passages into the lungs. As is well known in the art, ACE inhibitors have some cross reactivity with butylcholinesterase inhibitors. Butylcholinesterases are a structurally-related enzyme family, but have very different biological functions. Whereas inhibition of acetylcholinesterase can lead to beneficial accumulation of acetylcholine in synapses, inhibition of butylcholinesterase can result in respiratory failure, especially when the inhibitor enters the lung. This toxicity forms the basis for the widely known use of butylcholinesterase inhibitors as poisons and insecticides.

In more detailed embodiments of the invention, the methods and compositions employing nasal administration of ACE inhibitors for treating CNS disease are specifically designed to restrict contact of the formulation to the nasal turbinates and oropharynx. For intranasal solutions, droplet size of sprays are larger than about 20 to 100 μm, so that the spray droplets immediately drop to the nasal mucosa and do not enter the lungs as an aerosol. While a few droplets potentially can escape and enter the oropharynx, little or no drug will enter the lungs in the form of an aerosol. Optionally, gel formulations comprising one or more ACE inhibitor(s) are applied to the nasal mucosa, for example using pump or squeeze devices, and these methods also preclude substantial amounts of the ACE inhibitor from entering the lungs. Targeted delivery to the nasal mucosa may also involve limiting the dose volume, for example to less than about 1.0 ml per nostril, and in certain embodiments to approximately less than or equal to 0.2 ml per nostril, or less than or equal to about 0.1 ml per nostril. The intranasal formulations of the invention do not lead to toxic poisoning or other adverse side effects potentially associated with oral or pulmonary delivery of ACE inhibitors.

In certain embodiments, the ACE inhibitor is administered to the mammal in an effective dose of between about 0.1 mg to about 100 mg per dose, and up to 6 doses per day, more preferentially about 1 to 50 mg per dose, and most preferentially 1.5 to 12 mg per dose. Dosage is given preferably once per day, but acceptably four times per day or more. Dosing may have to be gradually increased to develop tolerance. The dosage regime is expected to be dependent on the degree and severity of symptoms, body weight, the presence or absence of renal failure or cirrhosis, and other factors that may be evaluated by the attending physician or veterinarian, and may vary widely.

In other detailed embodiments, the pharmaceutical compositions and delivery methods of the invention yield targeted CNS deliver. For example, in various embodiments a peak concentration of the ACE inhibitor in a CNS tissue or fluid of a subject following administration that is at least 10%, 15%, 20%, 25%, 30%, 35%, or 40% greater than a plasma concentration of the ACE inhibitor in a blood plasma of the same subject at the same time point after the drug is administered.

As used herein, the following definitions are provided as an aid in interpreting the claims and specification herein. Where works are cited by reference, and definitions contained therein are inconsistent with those supplied here, the definition used therein shall apply only to the work cited and shall not be applied to this disclosure.

“Mammal” shall include any of a class of warm-blooded higher vertebrates that nourish their young with milk secreted by mammary glands and have skin usually more or less covered with hair, and non-exclusively includes humans and non-human primates, their children, including neonates and adolescents, both male and female, livestock species, such as horses, cattle, sheep, and goats, and research and domestic species, including dogs, cats, mice, rats, guinea pigs, and rabbits. “Patient” or “subject” is used herein interchangeably with “mammal.”

“Dementia” shall mean a broad deterioration of intellectual functioning with impaired or absence of clarity in conscious awareness, and is characterized by one or more symptoms of impaired short term memory, impaired judgment, impaired rational intellect, and/or disorientation with respect to place or time. Dementia is considered “irreversible” when, as is typical, accompanied by organic brain disease. Dementia is always associated with disability in the conduct of an independent lifestyle. The symptoms of dementia encompass but are worse than those of cognitive impairment.

“Cognitive impairment” is a disorder in memory, problem solving, abstract reasoning and orientation that weakens an individual's ability to maintain an independent lifestyle. Mild cognitive impairment does not rise to the level of Alzheimer's disease and is not unusual in ageing. A hallmark is memory impairment with resulting confusion in the conduct of daily affairs.

“Intranasal delivery” shall mean delivery of a drug primarily via the mucosa of the nasal cavity. This includes the superior, middle and inferior nasal turbinates and the nasal pharynx. Note that the olfactory region is concentrated in the superior (upper ⅓) of the nasal turbinates. Cilial action pushes material back toward the oropharynx, so material deposited in the nasal vestibule encounters the nasal mucosa before entering the throat.

“Acetylcholinesterase inhibitor” shall mean a xenogenic or naturally-occurring compound that increases the concentration of acetylcholine through reversible inhibition of its hydrolysis by acetylcholinesterase.

“Native neurobiomolecule” refers to any cellular or humoral signal molecule that is genetically encoded for by a mammal or is formed in the normal metabolism of a mammal, and is found in normal mammals in the brain or CSF. By way of example, native neurobiomolecules include ganglioside, phosphatidylserine, brain-derived neurotropic factor, fibroblast growth factor, insulin, insulin-like growth factors, ciliary neurotropic factor, glia-derived nexin, cholinergic enhancing factors, phosphoethanolamine, and thyroid hormone T3. It is thought that lipids such as phosphatidylserine are components of a vesicular transport mechanism specific for these molecules. In contrast, ACE inhibitors for use within the current invention are typically selected from synthetic, non-naturally occurring chemicals herein termed “xenogenic molecules” or “xenogenic ACE inhibitors”.

“Xenogenic” refers herein to any synthetic product of man's chemical art and skills which is not found in nature as the natural product of a biosynthetic pathway of a mammal.

“Naturally occurring” refers to biomolecules that are extracted or derivatized from plant or animal sources, including oleagenous and petroleum deposits.

“Inhalation route”: As described in Remington's, “Inhalation may be employed for delivering gaseous or volatile substances into the systemic circulation, as with most general anesthetics. Absorption is virtually as rapid as the drug can be delivered into the alveoli of the lungs, since the alveolar and vascular epithelial membranes are quite permeable, blood flow is abundant and there is a very large surface area for absorption. Aerosols of nonvolatile substances also may be administered by inhalation, but the route is used infrequently for delivery into the systemic circulation because of various factors that contribute to erratic and difficult-to-achieve blood levels. Whether or not an aerosol reaches and is retained in the pulmonary alveoli depends critically upon particle size. Particles greater than 1 μm in diameter tend to settle in the bronchioles and bronchi, whereas particles less than 0.5 μm fail to settle and are mainly exhaled.” MR Franklin. “Drug Absorption, Action and Disposition” in Remington: The Science and Practice of pharmacy. 19^{th} ed. pp 711-12.

“Nasal route:” Drugs may be given intranasally by direct application to the nasal mucosa lining the nasal turbinates. The mucosa is richly vascularized and enervated and extends from the nasal nares to the upper boundary of the oropharynx and the lower boundaries of the sinus passages. Drugs applied to the nasal mucosa permeate trans-mucosally by either paracellular diffusion (passive), or by transcellular diffusion (passive or facilitated diffusion, or active transport). Passive diffusion is most conveniently employed for molecules less than 1 kilodaltons in size, perhaps up to 5 kilodaltons as a maximum for any substantial uptake. However, uptake by the nasal route of administration is not so limited. Nasal uptake of peptides and proteins of up to several hundred kilodaltons has been demonstrated in the past few years. Drug crossing the mucosal barrier enters nasal capillaries and the general circulation, bypassing the liver on the first pass. It is thought that drug may also enter the olfactory or trigeminal nerve bundle and be transported intra-axonally to the CNS. It is further speculated that drug may also diffuse into the cerebrospinal fluid (CSF) by the subarachnoid plexus and is transported in the flow of the CSF to other parts of the brain and spinal cord. Thus the nasal route of administration has multiple pathways and special relevance to drugs that target the CNS.

“Pharmaceutically acceptable” refers to a composition which, when administered to a human or a mammal by the indicated route of administration, provokes no adverse reaction which is unacceptably disproportionate to the benefit gained by administration of the compound. Although the therapeutic substance may be a source of adverse reaction, in some cases substitution of other excipients may modulate this toxicity.

“Delivery system” refers to a combination of excipients used to make pharmaceutically acceptable a formulation (with or without a device) used to deliver a measured dose or sufficient quantity of a drug by the chosen route of administration.

“Permeation-enhancing agent” refers to excipients in a composition for intranasal drug administration that enhance overall delivery, dose consistency and/or pharmaceutical acceptance. The methods for assessing enhancement are quantitative and are defined in the examples herein. Functional in vitro cell-based testing and methods are used to compare the effects of drug molecule by itself versus the same mass of drug molecule formulated and administered with excipients. “Enhancement” may be a multivariant value proposition encompassing one or more values or conditions of total bioavailability, net uptake, consistency of uptake and dosing, drug metabolism, degradation during dosing, drug targeting to active site(s), mucosal irritation, and overall safety and toxicity.

“About” is a relative term denoting an approximation of plus or minus 10% or 20% of the nominal value it refers to. For the field of pharmacology and clinical medicine and analogous arts that are the subject of this disclosure, this level of approximation is appropriate unless the value is specifically stated to be critical or to require a narrower range.

“Substantially free” refers to the level of a particular active ingredient in the compositions of the invention, wherein the particular active ingredient constitutes less than 20%, preferably less than 10%, more preferably less than 5%, and most preferably less than 1%, by weight based on the total weight of active ingredients in the composition.

“Degradant” refers to a product of a chemical reaction occurring in vitro during storage, dissolution, or upon stability testing under stress from heat, light, co-solutes and solvent, generally refers to a drug substance, and is contrasted with a metabolite.

“Metabolite” refers a product of a chemical or enzymatic reaction occurring in vivo, generally refers to a drug substance, and is contrasted with a degradant.

“Nasal mucosa” refers to the lining of the nasal cavity, where vascularized, and extending interiorly to the boundaries of the oropharynx and sinuses. It includes the superior turbinate, the middle turbinate, and the inferior turbinate. It is the latter two where most of the non-olfactory epithelium is found in man.

“Liquid” refers to a solution or formulation in the liquid state. Note that not all liquids are aqueous and that liquid behavior is not infrequently temperature dependent.

“Aqueous” refers to a solution formed in water, but may contain lesser amounts of other co-solvents. Note that not all aqueous solutions are liquids.

“Gel” refers to a thickened solution, aqueous or otherwise, used as a drug delivery vehicle and may be administered as a spray or an ointment when given intranasally.

“Short chain” refers to carbon lengths of C₁₂ or less, where the chain may be aliphatic or branched.

“Stability” or “stability during storage” refers to any compositional change measured in a parameter as a function of a commercially relevant time interval and conditions of storage, for example concentration, degradation, viscosity, pH, or particle size, which is greater than 10% over a time interval relevant to commercial shelf life, denotes instability. Changes less than or equal to 10% connote stability. The time period over which stability is measured is relative depending on the intended commercial distribution and storage conditions for the composition. “Accelerated stability testing” at higher temperature is sometimes taken as a more speedy way of extrapolating stability over longer periods of time. For example, a 4-month study at 40° C. can be taken to predict stability at controlled room temperature for over one year.

Three general modes of drug uptake and transport are contemplated herein:

“Paracellular” is used in its classical sense to indicate transport of a molecule between the cells of an epithelium, as in the nasal mucosa. Mannitol is taken as the reference permeant, and transport is passive, dependent on the size of the molecule and the size of the water channels in the cell junctions. This diameter is dependent on treatment of the membrane with enhancers that weaken the tight junctions or expand water channels.

“Transcellular” is used to indicate other forms of uptake at an epithelium that may be passive, facilitated diffusion, or active. This category includes diffusion of lipidic drugs in the membrane of epithelial cells from apical to basolateral sides, followed by escape of the drug by exchange or by blebbing into the cytosol. This category may include non-specific endocytosis and vesicular transport.

“Transaxonal” refers to specific uptake of native signaling molecules, defined here as neurobiomolecules, for tubulin-mediated active transport within nerve axons. This transport is typically vesicular and is believed to rely on receptor-mediated endocytosis. The phenomenon, first documented by Maitani in the rabbit nasal mucosa, is specialized for cytokines, hormones and the special lipid components that make up the vesicles formed for transport via this pathway.

The methods of the invention provide at least one ACE inhibitor to the CNS of a mammal, typically via intranasal delivery applying the ACE inhibitor to the nasal cavity of a mammal. Typical theraputic formulations in this context will comprise:
a. a liquid or gel solution (preferably an aqueous solution), or a powder formulation, containing at least one ACE inhibitor; and
b. at least one permeation-enhancing agent.

The methods of the invention that involve intranasal delivery improve the bioavailability of the ACE inhibitor to the CNS because the delivery route bypasses the digestive tract, liver and lungs.

Exemplary pharmaceutical compositions of the invention useful for the prevention and/or treatment of diseases and disorders of the CNS, include:
a. a liquid or gel solution, preferably an aqueous solution, of at least one ACE inhibitor; and
b. at least one permeation-enhancing agent;
   wherein the pharmaceutical composition is suitable for intranasal delivery.

The methods and compositions of the invention are suitable for preventing and/or treating diseases and disorders of the CNS, including, for example, neurological conditions associated with memory loss and cognitive impairment in mammals, including Parkinson's-type dementia, Huntington's-type dementia, Pick's-type dementia, CJ-type dementia, AIDS-related dementia, Lewy Body dementia, Rett's syndrome, epilepsy, brain malignancies or tumors, cognitive disorder associated with multiple sclerosis, Down's syndrome, progressive supranuclear palsy, certain forms of schizophrenia, depression, mania and related psychiatric conditions, Tourette's syndrome, mysasthenia gravis, attention deficit disorder, autism, dyslexia, forms of delirium, or dementia as a sequel to vascular stroke or cranial bleeding and brain injury, in their chronic, acute and relapsing forms, and nicotine withdrawal, among other CNS diseases and conditions treatable with ACE inhibitors.

Exemplary ACE inhibitors for use within the compositions and methods of the invention include xenogenic compounds such as donepezil, 6-O-desmethyl donezepil, tacrine, rivastigmine, neostigmine, pyrrdostigmine, physostigmine, ipidacrine, stacofylline, galantamine, galanthamine analogs, lycoramine, lycoramine analogs, physostigmine, ambenonium, demecarium, eprophonium, metrifonate, selegine, metrifonate, 3-[1-(phenylmethyl) piperidine-4-yl]-1-(2,3,4,5-tetrahydro-1H-1-benzazepine-8-yl)-1-propane, 5,7-dihydro-3-[2-(1-(phenylmethyl)-4-pyperidinyl)ethyl]-6H-pyrrolo-[4,5-f]-1,2-benzisoxazole-6-one, 4,4′-diaminodiphenylsulfone, tetrahydroisoquinolinyl carbamate of pyrroloindole derivative.

In addition, all pharmaceutically-acceptable salts and derivatives of the foregoing exemplary ACE inhibitors are contemplated to be useful within the methods and formulations provided herein. Pharmaceutically-acceptable salts include inorganic acid salts, organic amine salts, organic acid salts, alkaline earth metal salts and mixtures thereof. Suitable examples of pharmaceutically-acceptable salts include, but are not limited to, halide, glucosamine, alkyl glucosamine, sulfate, hydrochloride, carbonate, hydrobromide, N,N′-dibenzylethylene-diamine, triethanolamine, diethanolamine, trimethylamine, triethylamine, pyridine, picoline, dicyclohexylamine, phosphate, sulfate, sulfonate, benzoate, acetate, salicylate, lactate, tartate, citrate, mesylate, gluconate, tosylate, maleate, fumarate, stearate and mixtures thereof.

In certain preferred embodiments, the compositions and methods of the invention may further include a COX-2 inhibitor, huperzine (selegine) or 4,4′-diaminodiphenylsulfone.

In combination with the ACE inhibitor(s), the pharmaceutical compositions of the invention will often include at least one excipient. Authoritative reviews of the pharmaceutical arts with respect to intranasal formulations are provided by Behl and Chien (Behl, C R et al. 1998. “Effects of physicochemical properties and other factors on systemic nasal drug delivery,” Adv Drug Del Rev 29:89-116; Behl C R et al. 1998. “Optimization of systemic nasal drug delivery with pharmaceutical excipients,” Adv Drug Del Rev 29:117-133 and, Chien, Y W. 1992. 2^{nd} Ed. Novel Drug Delivery Systems, Marcel Dekker, NY).

The pharmaceutical compositions of the invention will also typically include at least one permeation-enhancing agent. As used herein, “permeation-enhancing agent” includes agents which enhance the release or solubility (e.g., from a formulation delivery vehicle), diffusion rate, bioavailability, penetration capacity and/or timing, uptake, residence time, stability, effective half-life, peak or sustained concentration levels, clearance, reduction of irritation, comfort, biotolerance, and other desired intranasal cavity delivery characteristics (e.g., as measured at the site of delivery, or at a selected target site of activity in the CNS) of the ACE inhibitor. Enhancement of intranasal delivery can thus occur by any of a variety of mechanisms, for example by increasing the diffusion, transport, persistence or stability of the ACE inhibitor, increasing membrane fluidity in the epithelium, increasing fluidity of mucous secretions, modulating availability or action of calcium and other ions that regulate intracellular or paracellular permeation, solubilizing mucosal membrane components (e.g., lipids), changing non-protein and protein sulfhydryl levels in a nasal mucosal layer, increasing water flux across the nasal mucosal surface, modulating epithelial junctional physiology, reducing nasal mucociliary clearance rates, and other mechanisms.

Suitable permeation-enhancing agents for use within the methods and compositions of the invention will generally be selected from one or more of the following:
a. an aggregation inhibitory agent;
b. a charge modifying agent;
c. a pH control or pH buffering system;
d. a redox control or redox ‘buffering’ system
e. a degradative enzyme inhibitory agent;
f. a mucolytic or mucus clearing agent;
g. a ciliostatic agent;
h. an absorption enhancing agent or system selected from the group consisting of:
   (i) a surfactant;
   (ii) a bile salt;
   (iii) a phospholipid additive, mixed micelle, liposome, or carrier system;
   (iv) an alcohol;
   (v) an enamine;
   (vi) a nitric oxide donor compound;
   (vii) a long-chain amphipathic molecule;
   (viii) a small hydrophobic uptake enhancer;
   (ix) sodium or a salicylic acid derivative;
   (x) a glycerol ester of acetoacetic acid;
   (xi) acyclodextrin or β-cyclodextrin derivative;
   (xii) a medium-chain or short-chain fatty acid;
   (xiii) a chelating agent;
   (xiv) an amino acid or salt thereof;
   (xv) an N-acetylamino acid or salt thereof;
   (xvi) an enzyme degradative to a selected membrane component;
   (xvii) an inhibitor of fatty acid synthesis; and
   (xvii) an inhibitor of cholesterol synthesis;
(i) a modulatory agent of epithelial junction physiology;
(j) a vasodilator agent;
(k) a stabilizing delivery vehicle, carrier, support or complex-forming species with which the acetylACE inhibitor is effectively combined, associated, contained, encapsulated or bound resulting in complexing or stabilization of said ACE inhibitor for enhanced intranasal delivery;
(l) a humectant or other anti-irritant; and
(m) a permeabilizing peptide agent.

Aggregation inhibitory agents include, among others, surfactants, salts such as NaCl, KCl, and sugars, particularly poloxamers that limit close approach of particles or reduce zeta potential between charged elements that would otherwise flocculate.

pH adjustment is typically achived using a TAC grade reagent NaOH or HCl. When buffering capacity is desired, a buffering system having a pK near the desired pH is selected. Buffering systems well accepted for topical pharmaceuticals include acetate, citrate, phosphate (at 4 and 7), imidazole, histidine, glycine, tartrate and TEA. Acids for pH adjustment and salt formation include hydrochloric acid, hydrobromic acid, hydriodic acid, sulfuric acid, carbonic acid, nitric acid, boric acid, phosphoric acid, acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and mixtures thereof. Bases for pH adjustment and salt formation include basic amino acids, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, diethanolamine, triethanolamine, triethylamine, and triisopropanolamine.

Redox control and redox “buffering” agents include ascorbic acid, ascorbyl palmitate, any of the tocopherols, alpha, beta, gamma, delta, and mixed tocopherols, and the corresponding tocotrienols,

Degradative enzyme inhibitory agent include PMSF, amastatin, bestatin, trypsin inhibitor, camostat mesilate, and boroleucine. P-aminobenzamidine, FK-448, camostat mesylate, sodium glycocholate, an amino acid, a modified amino acid, a peptide, a modified peptide, a polypeptide protease inhibitor, a complexing agent, a mucoadhesive polymer, a polymer-inhibitor conjugate, or a mixture thereof, aminoboronic acid derivatives, n-acetylcysteine, bacitracin, phosphinic acid dipeptide derivatives, pepstatin, antipain, leupeptin, chymostatin, elastatin, bestatin, hosphoramindon, puromycin, cytochalasin potatocarboxy peptidase inhibitor, amastatin, protinin, Bowman-Birk inhibitor, soybean trypsin inhibitor, chicken egg white trypsin inhibitor, chicken ovoinhibitor, human pancreatic trypsin inhibitor, EDTA, EGTA, 1,10-phenanthroline, hydroxychinoline, polyacrylate derivatives, chitosan, cellulosics, chitosan-EDTA, chitosan-EDTA-antipain, polyacrylic acid-bacitracin, carboxymethyl cellulose-pepstatin, polyacrylic acid-Bowman-Birk inhibitor, and mixtures thereof are also contemplated as enzyme inhibitory substances. (See, Bernkop-Schnurch, “The use of inhibitory agents to overcome the enzymatic barrier to perorally administered therapeutic peptides and proteins,” Journal of Controlled Release, 52:1-16).

Ciliostatic agents include preservatives such as benzalkonium chloride, EDTA, and surfactants such as bile salts, betaine, and quaternary ammonium salts.

Mucolytic agents include dithiothreitol, cysteine, methionine, threonine, s-adenosylmethionine.

Absorption enhancing agents are selected from the groups consisting of bile acids, bile salts, ionic, nonionic zwitterionic, anionic, cationic, gemini pair surfactants, phospholipids, alcohols, glycyrrhetinic acid and its derivatives, enamines, salicylic acid and sodium salicylate, acetoacetate glycerol esters, dimethylsulfoxide, n-methylpyrrolinidinone, cyclodextrins, medium chain fatty acids, short chain fatty acids, short and medium chain diglycerides, short and medium chain monoglycerides, short chain triglycerides, calcium chelators, amino acids, cationic amino acids, homopolymeric peptides, cationic peptides, n-acetylamino acids and their salts, degradative enzymes, fatty acid synthesis inhibitors, cholesterol synthesis inhibitors.

Absorption enhancers are screened on a case-by-case basis to determine the most suitable candidate. Various models have been studied, for example those of LeCluyse and Sutton (1997. “In vitro models for selection of development candidates. Permeability studies to define mechanisms of absorption enhancement” Advanced Drug Delivery Reviews, 23:163-183). In vitro methods with the EpiAirway model have proven to be valuable.

Absorption enhancers include PEG-fatty acid esters having useful surfactant properties. Among the PEG-fatty acid monoesters, esters of lauric acid, oleic acid, and stearic acid are especially useful. Surfactants include PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate. Polyethylene glycol (PEG) fatty acid diesters are also suitable for use as surfactants in nasal formulations. Hydrophilic surfactants include PEG-20 dilaurate, PEG-20 dioleate, PEG-20 distearate, PEG-32 dilaurate and PEG-32 dioleate. In general, mixtures of surfactants are also useful in the present invention, including mixtures of two or more commercial surfactant products. Several PEG-fatty acid esters are marketed commercially as mixtures or mono- and diesters. Suitable PEG glycerol fatty acid esters are PEG-20 glyceryl laurate, PEG-30 glyceryl laurate, PEG-40 glyceryl laurate, PEG-20 glyceryl oleate, and PEG-30 glyceryl oleate.

The reaction of alcohols or polyalcohols with a variety of natural and/or hydrogenated oil yields a large number of surfactants of different degrees of hydrophobicity or hydrophilicity. Commonly, the oils used are castor oil or hydrogenated castor oil, or an edible vegetable oil such as corn oil, olive oil, peanut oil, palm kernel oil, apricot kernel oil, or almond oil. Alcohols include glycerol, propylene glycol, ethylene glycol, polyethylene glycol, maltol, sorbitol, and pentaerythritol. Among these alcohol-oil transesterified surfactants, hydrophilic surfactants are PEG-35 castor oil (Incrocas-35), PEG-40 hydrogenated castor oil (Cremophor® RH 40), PEG-25 trioleate (TAGAT® TO), PEG-60 corn glycerides (Crovol® M70), PEG-60 almond oil (Crovol A70), PEG-40 palm kernel oil (Crovol PK70), PEG-50 castor oil (Emalex C-50), PEG-50 hydrogenated castor oil (Emalex® HC-50), PEG-8 caprylic/capric glycerides (Labrasol®), and PEG-6 caprylic/capric glycerides (Softigen® 767). Hydrophobic surfactants in this class include PEG-5 hydrogenated castor oil, PEG-7 hydrogenated castor oil, PEG-9 hydrogenated castor oil, PEG-6 corn oil (Labrafil 2125 CS), PEG-6 almond oil (Labrafil® M 1966 CS), PEG-6 apricot kernel oil (Labrafil 1944 CS), PEG-6 olive oil (Labrafil® M 1980 CS), PEG-6 peanut oil (Labrafil 1969 CS), PEG-6 hydrogenated palm kernel oil (Labrafil 2130 BS), PEG-6 palm kernel oil (Labrafil.2130 CS), PEG-6 triolein (Labrafil 2735 CS), PEG-8 corn oil (Labrafil WL 2609 BS), PEG-20 corn glycerides (Crovol M40), and PEG-20 almond glycerides (Crovol A40). The latter two surfactants are reported to have HLB values of about 10, which is the approximate border line between hydrophilic and hydrophobic surfactants (8 to 12 HLB). Derivatives of vitamins A, D, E, K, such as tocopheryl PEG-1000 succinate (TPGS, available from Eastman), are also suitable surfactants.

Polyglycerol esters of fatty acids are also suitable surfactants for the present invention. Among the polyglyceryl fatty acid esters, hydrophobic surfactants include polyglyceryl oleate (Plurol Oleique®), polyglyceryl-2 dioleate (Nikkol DGDO), and polyglyceryl-10 trioleate. Preferred hydrophilic surfactants include polyglyceryl-10 laurate (Nikkol Decaglyn® 1-L), polyglyceryl-10 oleate (Nikkol Decaglyn 1-O), and polyglyceryl-10 mono, dioleate (Caprol® PEG 860). Polyglyceryl polyricinoleates (Polymuls) are also preferred hydrophilic and hydrophobic surfactants. Hydrophobic surfactants include propylene glycol monolaurate (Lauroglycol® FCC), propylene glycol ricinoleate (Propymuls®), propylene glycol monooleate (Myverol® P-06), propylene glycol dicaprylate/dicaprate (Captex® 200), and propylene glycol dioctanoate (Captex® 800). Included are both mono- and diesters of propylene glycol. Mixtures of propylene glycol fatty acid esters and glycerol fatty acid esters are commercially available. One such mixture is composed of the oleic acid esters of propylene glycol and glycerol (Arlacel® 186). Another class of surfactants is the class of mono- and diglycerides. These surfactants are not always hydrophobic, depending on aliphatic chain length. Surfactants in this class of compounds include glyceryl monooleate (Peceol®), glyceryl ricinoleate, glyceryl laurate, glyceryl dilaurate (Capmul® GDL), glyceryl dioleate (Capmul® GDO), glyceryl mono/dioleate (Capmul® GMO-K), glyceryl caprylate/caprate (Capmul® MCM), caprylic acid mono/diglycerides (Imwitor® 988), and mono- and diacetylated monoglycerides (Myvacet® 9-45), functioning well as absoption enhancers. Sterols and derivatives of sterols have some use in the present invention. A hydrophobic surfactant in this class is PEG-24 cholesterol ether (Solulan® C-24).

A variety of PEG-sorbitan fatty acid esters are available. In general, these surfactants are hydrophilic, although several hydrophobic surfactants of this class can be used. Among the PEG-sorbitan fatty acid esters, hydrophilic surfactants include PEG-20 sorbitan monolaurate (Tween-20), PEG-20 sorbitan monopalmitate (Tween-40), PEG-20 sorbitan monostearate (Tween-60), and PEG-20 sorbitan monooleate (Tween-80).

Ethers of polyethylene glycol and alkyl alcohols are also useful as surfactants. Hydrophobic ethers include PEG-3 oleyl ether (Volpo 3) and PEG-4 lauryl ether (Brij 30). Several hydrophilic PEG-alkyl phenol surfactants are available, and are suitable for use in nasal compositions for drug delivery.

The POE-POP block copolymers are a unique class of polymeric surfactants. The unique structure of the surfactants, with hydrophilic POE and hydrophobic POP moieties in well-defined ratios and positions, provides a wide variety of surfactants suitable for use in the present invention. These surfactants are available under various trade names, including Synperonic PE series (ICI); Pluronic® series (BASF), Emkalyx, Lutrol (BASF), Supronic, Monolan, Pluracare, and Plurodac®. The generic term for these familiar polymers is “poloxamer.” Hydrophilic surfactants of this class include Poloxamers 108, 188, 217, 238, 288, 338, and 407. Hydrophobic surfactants in this class include Poloxamers 124, 182, 183, 212, 331, and 335. Surfactants of this class are commonly known as poloxamers and tetronics.

Sorbitan esters of fatty acids are suitable surfactants for use in the present invention. Among these esters, preferred hydrophobic surfactants include sorbitan monolaurate (Arlacel 20), sorbitan monopalmitate (Span-40), sorbitan monooleate (Span-80), sorbitan monostearate, and sorbitan tristearate. Esters of lower alcohols (C₂ and C₄) and fatty acids (C₈ to C₁₈) are suitable surfactants for use in the present invention. Among these esters, hydrophobic surfactants include ethyl oleate (Crodamol® EO), isopropyl myristate (Crodamol IPM), and isopropyl palmitate (Crodamol IPP).

Ionic surfactants, including cationic, anionic and zwitterionic surfactants, are suitable hydrophilic surfactants for use in the present invention. Anionic surfactants include fatty acid salts and bile salts. Cationic surfactants include camitines such as camityl palmitate. Specifically, preferred ionic surfactants include sodium oleate, sodium lauryl sulfate, sodium lauryl sarcosinate, sodium dioctyl sulfosuccinate, sodium cholate, sodium taurocholate; lauroyl carnitine; palmitoyl carnitine; and myristoyl carnitine. It will be appreciated by a skilled formulator, that any pharmaceutically acceptable counterion may be used. Unlike typical non-ionic surfactants, these ionic surfactants are generally available as pure compounds rather than proprietary mixtures. These compounds are readily available from a variety of suppliers such as Aldrich, Sigma, and the like.

Particular examples of surfactants that are pH dependent include free fatty acids, particularly C₆ to C₂₂ fatty acids, and the bile acids. Ionizable surfactants include fatty acids and their salts, such as caprylic acid/sodium caprylate, oleic acid/sodium oleate, capric acid/sodium caprate; ricinoleic acid/sodium ricinoleate, linoleic acid/sodium linoleate, and lauric acid/sodium laurate; trihydroxy bile acids and their salts, such as cholic acid (natural), glycocholic acid and taurocholic acid; dihydroxy bile acids and their salts, such as deoxycholic acid (natural), glycodeoxycholic acid, taurodeoxycholic acid, chenodeoxycholic acid (natural), glycochenodeoxycholic acid, taurochenodeoxycholic acid, ursodeoxycholic acid, tauroursodeoxycholic acid, and glycoursodeoxycholic acid; monohydroxy bile acids and their salts, such as lithocholic acid (natural); sulfated bile salt derivatives; sarchocholate; fusidic acid and its derivatives; phospholipids, such as phosphatidyl choline, phosphatidyl ethanolamine, phosphatidylinositol, lysolecithin, and palmitoyl lysophosphatidyl choline; carnitines, such as palmitoyl carnitine, lauroyl carnitine and myristoyl carnitine; cyclodextrins, including alpha, beta and gamma cyclodextrins and their chemically substituted derivatives such as hydroxy propyl, 2-hydroxypropyl-β-cyclodextrin and heptakis(2,6-di-O-methyl-β-cyclodextrin and sulfobutyl ether are included here.

Ionic surfactants include the ionized form of alkyl ammonium salts; bile acids and salts, analogues, and derivatives thereof; fusidic acid and derivatives thereof; fatty acid derivatives of amino acids, oligopeptides, and polypeptides; glyceride derivatives of amino acids, oligopeptides, and polypeptides; acyl lactylates; mono-,diacetylated tartaric acid esters of mono-,diglycerides; succinylated monoglycerides; citric acid esters of mono-,diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; carnitines; and mixtures thereof.

Further included are the ionized form of bile acids and salts, analogues, and derivatives thereof; lecithins, lysolecithin, phospholipids, lysophospholipids and derivatives thereof; salts of alkylsulfates; salts of fatty acids; sodium docusate; acyl lactylates; mono and diacetylated tartaric acid esters of mono-,diglycerides, succinylated monoglycerides; citric acid esters of mono- and diglycerides; carnitines; and mixtures thereof. Further embodiments include PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof. Useful surfactants are the ionized forms of lecithin, lysolecithin, phosphatidylcholine, phosphatidylethanolamine, phosphatidylglycerol, lysophosphatidylcholine, PEG-phosphatidylethanolamine, lactylie esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, glycodeoxycholate, cholylsarcosine, caproate, caprylate, caprate, laurate, oleate, lauryl sulfate, docusate, and salts and mixtures thereof, with the most preferred ionic surfactants being lecithin, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, taurocholate, caprylate, caprate, oleate, lauryl sulfate, docusate, and salts and mixtures thereof.

Surfactants can also be formed from alcohols; for example polyoxyethylene alkylethers; fatty acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid derivatives of mono/diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; polyoxyethylene hydrogenated vegetable oils; and the un-ionized (neutral) forms of ionizable surfactants. Hydrophobic surfactants can be reaction mixtures of polyols and fatty acids, glycerides, vegetable oils, hydrogenated vegetable oils, and sterols. The hydrophobic surfactant can be selected from the group consisting of fatty acids; lower alcohol fatty acid esters; polyethylene glycol glycerol fatty acid esters; polypropylene glycol fatty acid esters; polyoxyethylene glycerides; glycerol fatty acid esters; acetylated glycerol fatty acid esters; lactic acid derivatives of mono/diglycerides; sorbitan fatty acid esters; polyoxyethylene sorbitan fatty acid esters; polyoxyethylene-polyoxypropylene block copolymers; polyoxyethylene vegetable oils; polyoxyethylene hydrogenated vegetable oils; and reaction mixtures of polyols and fatty acids, glycerides, vegetable oils, hydrogenated vegetable oils, and sterols. Lower alcohol fatty acids esters; polypropylene glycol fatty acid esters; propylene glycol fatty acid esters; glycerol fatty acid esters; acetylated glycerol fatty acid esters; lactic acid derivatives of mono/diglycerides; sorbitan fatty acid esters; polyoxyethylene vegetable oils; and mixtures thereof, with glycerol fatty acid esters and acetylated glycerol fatty acid esters are contemplated. Among the glycerol fatty acid esters, the esters comprise mono- or diglycerides, or mixtures of mono- and diglycerides, where the fatty acid moiety is a C₆ to C₂₂ fatty acid.

Also included are hydrophobic surfactants which are the reaction mixture of polyols and fatty acids, glycerides, vegetable oils, hydrogenated vegetable oils, and sterols. Polyols are polyethylene glycol, sorbitol, propylene glycol, and pentaerythritol.

Modulators of tight junction permeability include, among others, EDTA, calcium complexing agents, citric acid, salicylates, n-acyl derivatives of collagen, enamines and permeabilizing peptides as described herein.

Bioadhesives include chitosan, carboxymethylcellulose, carbopol, polycarbophil, hydroxy propyl methyl cellulose, tragacanth gum and others.

Vasodilators such as nitrous oxide (NO), nitroglycerin, and arginine are included to increase blood flow in the nasal capillary bed. These include S-nitroso-N-acetyl-DL-penicillamine, NOR1, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate.

Stabilizing delivery vehicles, carriers, support or complex-forming species include cyclodextrins, EDTA, microencapsulation systems, and liposomal formulations such as the bisphere and biosome technologies (U.S. Pat. No. 5,665,379).

Humectant or other anti-irritants are selected from compounds such as glycerol, 1,3 butanediol, tocopherol, petroleum, mineral oil, micro-crystalline waxes, polyalkenes, paraffin, cerasin, ozokerite, polyethylene, perhydrosqualene, dimethicones, cyclomethicones, alkyl siloxanes, polymethylsiloxanes, methylphenylpolysiloxanes, hydroxylated milk glyceride, castor oil, soy bean oil, maleated soy bean oil, safflower oil, cotton seed oil, corn oil, walnut oil, peanut oil, olive oil, cod liver oil, almond oil, avocado oil, palm oil, sesame oil, liquid sucrose octaesters, blends of liquid sucrose octaesters and solid polyol polyesters, lanolin oil, lanolin wax, lanolin alcohol, lanolin fatty acid, isopropyl lanolate, acetylated lanolin, acetylated lanolin alcohols, lanolin alcohol linoleate, lanolin alcohol riconoleate, beeswax, beeswax derivatives, spermaceti, myristyl myristate, stearyl stearate, carnauba and candelilla waxes, cholesterol, cholesterol fatty acid esters and homologs thereof, lecithin and derivatives, sphingolipids, ceramides, glycosphingo lipids and homologs thereof. Sodium pyroglutamate, hyaluronic acid, chitosan derivatives (carboxymethyl chitin), β-glycerophosphate, lctamide, acetamide, ethyl, sodium and triethanolamine lactates, metal pyrrolidonecarboxylates (especially of Mg, Zn, Fe or Ca), thiamorpholinone, orotic acid, C₃-C₂₀ alpha-hydroxylated carboxylic acids, in particular α-hydroxypropionic acid, polyols, in particular inositol, glycerol, diglycerol, sorbitol, saccharide polyols, in particular alginate and guar, proteins, in particular soluble collagen and gelatin, lipoprotides chosen from mono- or polyacylated derivatives of amino acids or of polypeptides in which the acid residue RCO contains a C₁₃-C₁₉ hydrocarbon chain, in particular palmitoylcaseinic acid, palmitoylcollagenic acid, the O,N-dipalmitoyl derivative of hydroxyproline, sodium stearoylglutamate, the stearoyl tripeptide of collagen, the oleyltetra- and pentapeptide of collagen, hydroxyprolin, linoleate, uea and its derivatives, in particular xanthyl urea, cutaneous tissue extract, in particular that marketed by Laboratoires Serobiologiques de Nancy (LSN) under the name “OSMODYN®”, containing peptides, amino acids and saccharides and 17% of mannitol. A combination of glycerol, urea and palmitoylcaseinic acid is useful.

Thickeners include methylcellulose, polyvinylpyrrolidone, hydroxycellulose, chitin, sodium alginate, xanthan gum, quince seed extract, tragacanth gum, starch and the like, semi-synthetic polymeric materials such as cellulose ethers (e.g. hydroxyethyl cellulose, methyl cellulose, carboxymethyl cellulose, hydroxy propylmethyl cellulose), polyvinylpyrrolidone, polyvinylalcohol, guar gum, hydroxypropyl guar gum, soluble starch, cationic celluloses, cationic guards and the like and synthetic polymeric materials such as carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers polacrylates; fumed silica natural and synthetic waxes, alkyl silicone waxes such as behenyl silicone wax; aluminum silicate; lanolin derivatives such as lanesterol; higher fatty alcohols; polyethylenecopolymers; narogel; polyammonium stearate; sucrose esters; hydrophobic clays; petroleum; and hydrotalcites.

In one embodiment, the permeation-enhancing agents are selected from citric acid, sodium citrate, propylene glycol, glycerin, L-ascorbic acid, sodium metabisulfite, EDTA disodium, benzalkonium chloride and sodium hydroxide.

Preferably, the pharmaceutical composition of the invention are substantially free of native neurobiomolecules, including ganglioside, phosphatidylserine, brain-derived neurotropic factor, fibroblast growth factor, insulin, insulin-like growth factors, ciliary neurotropic factor, glia-derived nexin, cholinergic enhancing factors, phosphoethanolamine and thyroid hormone T3.

Within certain aspects of the invention, absorption-promoting agents for coordinate administration or combinatorial formulation with ACE inhibitors are selected from small hydrophilic molecules, including but not limited to, dimethyl sulfoxide (DMSO), dimethylformamide, ethanol, propylene glycol, 1,3 butanediol, and the 2-pyrrolidones. Alternatively, long-chain amphipathic molecules, for example, deacylmethyl sulfoxide, azone, sodium laurylsulfate, oleic acid, and the bile salts, may be employed to enhance mucosal penetration of the ACE inhibitor. In additional aspects, surfactants (e.g., polysorbates) are employed as adjunct compounds, processing agents, or formulation additives to enhance intranasal delivery of the ACE inhibitor. These penetration enhancing agents typically interact at either the polar head groups or the hydrophilic tail regions of molecules which comprise the lipid bilayer of epithelial cells lining the nasal mucosa (Barry, Pharmacology of the Skin, Vol. 1, pp. 121-137, Shroot et al., Eds., Karger, Basel, 1987; and Barry, J. Controlled Release 6:85-97, 1987, each incorporated herein by reference). Interaction at these sites may have the effect of disrupting the packing of the lipid molecules, increasing the fluidity of the bilayer, and facilitating transport of the drug across the mucosal barrier. Interaction of these penetration enhancers with the polar head groups may also cause or permit the hydrophilic regions of adjacent bilayers to take up more water and move apart, thus opening the paracellular pathway to transport of the ACE inhibitor. In addition to these effects, certain enhancers may have direct effects on the bulk properties of the aqueous regions of the nasal mucosa. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the ACE inhibitor from the vehicle into the mucosa.

Additional permeation-enhancing agents that are useful within the coordinate administration and processing methods and combinatorial formulations of the invention include, but are not limited to, mixed micelles; enamines; nitric oxide donors (e.g., S-nitroso-N-acetyl-DL-penicillamine, NOR1, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion, paracellular or intra- or trans-epithelial absorption-promoting agents that are physiologically compatible for mucosal delivery.

Other permeation-enhancing agents are selected from a variety of carriers, bases and excipients that enhance intranasal delivery, stability, activity or paracellular or trans-epithelial uptake of the ACE inhibitor. These include, inter alia, cyclodextrins and β-cyclodextrin derivatives (e.g., 2-hydroxypropyl-p-cyclodextrin and heptakis(2,6-di-O-methyl-β-cyclodextrin). These compounds, optionally conjugated with one or more of the active ingredients and further optionally formulated in an oleaginous base, enhance bioavailability in the pharmaceutical compositions of the invention. Yet additional permeation-enhancing agents adapted for mucosal delivery include medium-chain fatty acids, including mono- and diglycerides (e.g., sodium caprate, extracts of coconut oil, Capmul), and triglycerides (e.g., amylodextrin, Estaram 299, Miglyol 810).

The compositions of the present invention may be supplemented with any suitable permeation enhancement agent that facilitates absorption, diffusion, or penetration of ACE inhibitor across nasal mucosal barriers. The permeation enhancement may be any agent or system that is pharmaceutically acceptable. Thus, in more detailed aspects of the invention compositions are provided that incorporate one or more penetration-promoting agents selected from sodium salicylate and salicylic acid derivatives (acetyl salicylate, choline salicylate, salicylamide, etc.); amino acids and salts thereof (e.g. monoaminocarboxlic acids such as glycine, alanine, phenylalanine, proline, hydroxyproline, etc.; hydroxyamino acids such as serine; acidic amino acids such as aspartic acid, glutamic acid, etc; and basic amino acids such as lysine, arginine eto—inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts), polyamino acids, and polycationic polymers. Also provided as uptake enhancers within the methods and compositions of the invention are substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

Permeabilizing peptides for use within the invention include any peptide that increases transmucosal delivery of drugs, for instance nucleic acid, peptide, protein and/or small molecule drugs. Permeabilizing peptides for use within the invention often function by reversibly enhancing mucosal epithelial paracellular transport of drugs, for example by modulating epithelial junctional structure and/or physiology to render epithelial layers more permeable to paracellular drug transport. The activities of permeabilizing peptides often further include reversible reduction of transepithelial electrical resistance (TEER).

Permeabilizing peptides for enhancing delivery of ACE inhibitors within the compositions and methods of the invention include, for example, any one or combination of the following peptides, or active fragments, conjugates, or complexes thereof:

(SEQ ID NO: 1)

RKKRRQRRRPPQCAAVALLPAVLLALLAP;

(SEQ ID NO: 2)

RQIKIWFQNRRMKWKK;

(SEQ ID NO: 3)

GWTLNSAGYLLGKJNLKALAALAKKIL;

(SEQ ID NO: 4)

KLALKLALKALKAALKLA;

(SEQ ID NO: 7)

KLWSAWPSLWSSLWKP;

(SEQ ID NO: 8)

AAVALLPAVLLALLAPRKKRRQRRRPPQ;

(SEQ ID NO: 9)

LLETLLKPFQCRJCMRNFSTRQARRNHRRRRRR;

(SEQ ID NO: 10)

RRRQRRKRGGDIMGEWGNEIFGAIAGFLG;

(SEQ ID NO: 11)

KETWWETWWTEWSQPGRKKRRQRRRPPQ;

(SEQ ID NO: 12)

GLGSLLKKAGKKLKQPKSKRKV;
and

(SEQ ID NO: 13)

KGSKKAVTKAQKKDGKKRKRSRKESYSVYVYKVLKQ

Permeation enhancing peptides useful within the invention are exemplified by the peptide KLALKLALKALKAALKLA (SEQ ID NO: 4), designated herein as “PN159”. PN159 enhances mucosal permeation for peptide therapeutic drugs, including parathyroid hormone (PTH) and Peptide YY. This permeation enhancing activity evinced for PN159 can be equivalent to, or greater than, epithelial permeation enhancement achieved through the use of one or multiple small molecule permeation enhancers. Thus, PN159 and other permeabilizing peptides can replace the role of small molecule permeation enhancers to facilitate mucosal delivery of ACE inhibitors.

PN159 features an unmodified N terminus. Other useful permeabilizing peptides include modified peptides related to PN159, such as a bromoacetate derivative of PN159 designated as “PN0068”. Like certain other permeabiizing peptides within the invention, PN159 and PN0068 have a multitude of cationic moieties such as lysine or arginine.

The permeabilizing peptides for use within the invention of the invention may include natural or synthetic peptides (comprised of two or more covalently linked amino acids), proteins, peptide or protein fragments, peptide or protein analogs, peptide or protein mimetics, and chemically modified derivatives or salts of active peptides or proteins. As used herein, the term “permeabilizing peptide” is intended to embrace all of these active species, i.e., peptides and proteins, peptide and protein fragments, peptide and protein analogs, peptide and protein mimetics, and chemically modified derivatives, conjugates, and salts of active peptides or proteins. Often, the permeabilizing peptides or proteins are muteins that are readily obtainable by partial substitution, addition, or deletion of amino acids within a naturally occurring or native (e.g., wild-type, naturally occurring mutant, or allelic variant) peptide or protein sequence. Additionally, active fragments of native peptides or proteins are included. Such mutant derivatives and fragments substantially retain the desired permeabilizing activity of the native peptide or proteins. In the case of peptides or proteins having carbohydrate chains, biologically active variants marked by alterations in these carbohydrate species are also included within the invention.

The permeabilizing peptides for use within the invention of the invention are formulated in a pharmaceutical composition comprising a permeabilizing effective amount of the permeabilizing peptide, protein, analog or mimetic sufficient to yield improved delivery of an ACE inhibitor to a targeted cell, tissue or compartment, typically in the CNS. The permeabilizing peptides and related compositions and methods of the invention usually function by reversibly enhancing mucosal epithelial paracellular transport, typically by modulating epithelial junctional structure and/or physiology at a mucosal epithelial surface in the subject.

Additional description pertaining to permeabilizing peptides useful within the instant invention is provided, for example, in Provisional U.S. Patent Application No. 60/612,121, filed by Cui et al. on Sep. 21, 2004, and in related Provisional United States Patent Application entitled “PERMEABILIZING PEPTIDES FOR ENHANCED MUCOSAL DELIVERY OF MACROMOLECULAR AND SMALL MOLECULE THERAPEUTIC COMPOUNDS” filed by Cui et al. on Apr. 1, 2005 and identified by Attorney Docket No. 04-15P2, each of which disclosures are incorporated herein by reference.

Delivery of ACE inhibitors across the nasal mucosal epithelium can occur by a predominantly “paracellular” pathway, although some transcellular transport of the more lipophilic compounds is likely. The extent to which either paracellular or transcellular uptake dominates the overall flux and bioavailability of a drug molecule depends not only on the size of the drug molecule and its physico-chemical properties and on the excipients in the formulation, and its physical state (solid, emulsion, gel, liquid), but also on the cellar response of the nasal mucosal epithelium. Paracellular transport involves only passive diffusion, and is especially important for hydrophilic molecules smaller than 1 kilodalton (Kda), whereas transcellular transport can occur by passive, facilitated or active processes following endocytosis or membrane fusion. Generally, hydrophilic, passively transported, polar solutes, particularly small-molecular weight xenogenic chemicals (i.e., those with MW<1 KDa), diffuse through the paracellular route, while native proteins, peptides and lipophilic solutes, including hydrophobic ACE inhibitors, can use in part or exclusively the transcellular route of transmucosal uptake.

The nasal mucosa consists of two tissue subdomains, the olfactory membrane and the non-olfactory domain. The olfactory epithelium has a distinctive layered columnar structure containing specialized olfactory receptor cells and supporting cell types. The non-olfactory membrane is highly vascularized and the surface covered by a ciliated layered columnar epithelium. The veins of the nasal cavity drain into the superior ophthalmic vein and facial vein, which are collected in the jugular vein for return to the heart. Native neurobiomolecules with specific receptors may gain access directly across the olfactory mucosa to the cranial nerves and undergo transaxonal transport to the CNS as proposed by Frey (U.S. Pat. No. 6,180,603) but this method is limited to the upper third of the nasal turbinates and to native neurobiomolecules which are recognized for transport. Alternatively, paracellular transport into the blood and CSF is possible through tight junctions in the non-olfactory membrane and transcellular transport is possible by nonspecific endocytosis, by perturbation of lipid membranes, and by cell mediated transcytosis. We differentiate here the paracellular and transcellular transport mechanisms from the specialized transaxonal transport mechanism described by Frey and by Maitani et al. (1986. “Intranasal administration of β-interferon in rabbits,” Drug Design Delivery 1:65-70). Olfactory epithelium is concentrated in the superior nasal turbinate. Non olfactory membrane, richly vascularized, dominates in the middle and inferior nasal turbinates.

Absorption and bioavailability for passively and actively absorbed solutes can be evaluated, in terms of the sum of the paracellular and transcellular delivery components, for any selected ACE inhibitor within the scope of the invention. The contributions of the pathways can be distinguished according to well known methods, such as in vitro epithelial cell culture permeability assays (See, e.g., Hilgers, et al., Pharm. Res. 7:902-910, 1990; Wilson et al., J. Controlled Release 11:25-40, 1990; Artursson. I., Pharm. Sci. 79:476-482, 1990; Cogburn et al., Pharm. Res. 8:210216, 1991; Pade et al., Pharmaceutical Research 14:1210-1215, 1997, each incorporated herein by reference with respect to the methodologies taught therein). However, it should be cautioned that clinical studies in man are needed before extrapolating drug uptake for therapy of a clinical condition.

For passively absorbed drugs, the relative contribution of paracellular and transcellular pathways to drug transport depends upon the pKa, lipophilicity as measured crudely by the partition coefficient, molecular radius and ionic charge(s) on the drug (molecular weight has some predictive value), the pH of the luminal environment in which the drug is delivered, the buffering capacity of the formulation, and the area of the absorbing surface. The paracellular route represents a relatively small fraction of accessible surface area of the nasal mucosal epithelium. In general terms, it has been reported that cell membranes occupy a mucosal surface area that is a thousand times greater than the area occupied by the paracellular spaces. Thus, the smaller accessible area, and the size- and charge-based discrimination against large (i.e., greater than 5 KDa) molecular permeation would suggest that the paracellular route could be a generally less favorable route than transcellular delivery for drug transport. However, and surprisingly, the methods and compositions of the present invention provide for significantly enhanced transport of ACE inhibitors into and across the non-olfactory nasal mucosal epithelia via the paracellular route, with surprising increases in bioavailability relative to oral administration, and increased targeting to the CNS.

The pharmaceutical compositions of the invention are specially formulated for nasal delivery. With nasal breathing, nearly all particles with a size of about 10-20 μm or larger are deposited on the nasal mucosa, whereas those less than 2 μm can pass through the nasal cavity and be deposited in the lungs. Formulations are optimized as to their physical state and chemical composition so as to be optimally suited for intranasal delivery. Nasal formulations may be, among others: powders gels, ointments, nose drops, tampons, sponges, and sprays. Powders are dispensed with special nasal applicators. Nose sprays are dispensed by a number of devices ranging from a simple squeeze bottle to a relatively complicated piston or pump. For aqueous formulations, the viscosity and interfacial tension determines in great measure the type of device that can be used for intranasal delivery with any particular formulation. For the non-aqueous liquid formulations contemplated herein, surface tension rarely is a factor and viscosity dominates in determining expelled droplet size. The pharmaceutical compositions of the invention may be applied to one or both nasal mucosal surfaces.

In other embodiments, a viscosifier or thickener, such as a gel polymer, may be incorporated into the formulations to increase droplet size and to ensure that the pharmaceutical compostion of the invention remains in the nose. Other approaches to retaining the drug bolus in the nose include use of a higher concentration of drug, and the use of low molecular weight polyoxyethylene glycol, propylene glycols, glycerol, 1,3-butanediol, or low MW mono- and diglycerides as rapid penetrants, in combination or singularly, essentially instantaneously hydrating the nasal mucosa and thereby anchoring the formulation in the mucous layer. Suitable aqueous sprays may be delivered in a coarse particulate or droplet form, on the order of 10 to 1000 μm diameter, so that the droplets go no farther than the nose. The applicator is inserted in the nasal vestibule and squeezed, a dose of the formulation usually no greater than 0.5-0.9 mL, often less than 0.2 mL, and in certain cases about 0.1 mL, is sprayed out and deposits itself on the walls of the nose, and is immediately fixed there by the non-aqueous solvents. Although a very small amount of material can enter the oropharynx before encountering the wall of the respiratory passage, little or essentially no material enters the lungs.

In more detailed aspects, the ACE inhibitor may be administered to the mammal in an effective dose of between about 0.1 mg and 100 mg.

In other detailed aspects, the pharmaceutical compositions of the invention may be formulated to possess a pH in an approximate range of about pH 3.0-6.0, pH 3.0-5.0, or pH 3.0-4.0.

In addition to the ACE inhibitor and permeation-enhancing agent, the pharmaceutical compositions of the invention may include a pharmaceutically acceptable carrier or vehicle. As used herein, “carrier” means a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, pp. 1857-1859, 1990, which is incorporated herein by reference. Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. The amount of active ingredient that can be combined with the carrier materials to produce a single dosage form will vary depending upon the particular mode of intranasal administration.

The pharmaceutical compositions of the invention are generally sterile and stable for pharmaceutical use. As used herein, “stable” means a formulation that fulfills all chemical and physical specifications with respect to identity, strength, quality, and purity which have been established according to the principles of Good Manufacturing Practice, as set forth by appropriate governmental regulatory bodies.

As used herein, a “mucosally effective amount of ACE inhibitor” contemplates effective mucosal delivery of ACE inhibitor to a target site for drug activity in the subject.

In various embodiments of the invention, the ACE inhibitor is combined with one, two, three, four or more of the permeation-enhancing agents recited in (a)-(m), above. These permeation-enhancing agents may be admixed, alone or together, with the ACE inhibitor, or otherwise combined therewith in a pharmaceutically acceptable formulation or delivery vehicle. Formulation of an ACE inhibitor with one or more of the permeation-enhancing agents according to the teachings herein (optionally including any combination of two or more delivery-enhancing agents selected from (a)-(m) above) provides for increased bioavailability of the ACE inhibitor following delivery thereof to a nasal mucosal surface of a mammal.

In related aspects of the invention, a variety of coordinate administration methods are provided for enhanced intranasal delivery of ACE inhibitors. These methods comprise the step, or steps, of administering to a mammal an effective amount of at least one ACE inhibitor in a coordinate administration protocol with one or more intranasal delivery-enhancing agents with which the ACE inhibitor(s) is/are effectively combined, associated, contained, encapsulated or bound to stabilize the active agent for enhanced intranasal delivery.

To practice a coordinate administration method according to the invention, any combination of one, two or more of the intranasal delivery-enhancing agents recited in (a)-(m), above, may be admixed or otherwise combined for simultaneous intranasal administration. Alternatively, any combination of one, two or more of the mucosal delivery-enhancing agents recited in (a)-(m) can be mucosally administered, collectively or individually, in a predetermined temporal sequence separated from mucosal administration of the ACE inhibitor (e.g., by pre-administering one or more of the delivery-enhancing agent(s)), and via the same or different delivery route as the ACE inhibitor (e.g., to the same or to a different mucosal surface as the ACE inhibitor, or even via a non-mucosal (e.g., intramuscular, subcutaneous, or intravenous route). Coordinate administration of the ACE inhibitor with any one, two or more of the delivery-enhancing agents according to the teachings herein provides for increased bioavailability of the ACE inhibitor following delivery thereof to a mucosal surface of a mammal.

In additional related aspects of the invention, various “multi-processing” or “co-processing” methods are provided for preparing formulations of ACE inhibitor for enhanced mucosal delivery. These methods include one or more processing or formulation steps wherein one or more ACE inhibitor(s) is/are serially, or simultaneously, contacted with, reacted with, or formulated with, one, two or more (including any combination of) of the permeation-enhancing agent.

To practice the multi-processing or co-processing methods according to the invention, the ACE inhibitor is/are exposed to, reacted with, or combinatorially formulated with any combination of one, two or more of the permeation-enhancing agents recited in (a)-(m), above, either in a series of processing or formulation steps, or in a simultaneous formulation procedure, that modifies the ACE inhibitor (or other formulation ingredient) in one or more structural or functional aspects, or otherwise enhances intranasal delivery of the active agent in one or more (including multiple, independent) aspect(s) that are each attributed, at least in part, to the contact, modifying action, or presence in a combinatorial formulation, of a specific intranasal delivery-enhancing agent recited in (a)-(m), above.

Many known reagents that are reported to enhance mucosal absorption also cause irritation or damage to mucosal tissues (see, e.g., Swenson and Curatolo, Adv. Drug Delivery Rev. 8:39-92, 1992, incorporated herein by reference). In this regard, the combinatorial formulation and coordinate administration methods of the present invention incorporate effective, minimally toxic delivery-enhancing agents to enhance intranasal delivery of ACE inhibitors useful within the invention.

While the mechanism of absorption promotion may vary with different permeation-enhancing agents of the invention, useful reagents in this context will not substantially adversely affect the mucosal tissue and will be selected according to the physicochemical characteristics of the particular ACE inhibitor or other active or delivery-enhancing agent. In this context, permeation-enhancing agents that increase penetration or permeability of mucosal tissues will often result in some alteration of the protective permeability barrier of the nasal mucosa. For such permeation-enhancing agents to be of value within the invention, it is generally desired that any significant changes in permeability of the nasal mucosa be reversible within a time frame appropriate to the desired duration of drug delivery. Furthermore, there should be no substantial, cumulative toxicity, nor any permanent deleterious changes induced in the barrier properties of the nasal mucosa with long-term use.

Within various aspects of the invention, improved mucosal delivery formulations and methods are provided which allow delivery of ACE inhibitors and other therapeutic agents within the invention across mucosal barriers between administration and selected target sites. Typically, the ACE inhibitor is efficiently loaded at effective concentration levels in a carrier or other delivery vehicle, and is delivered and maintained in a stabilized form, e.g., at the nasal mucosa and membranes, until delivered by facilitated or passive diffusion to a remote target site for drug action (e.g., the blood stream or CNS). The ACE inhibitor may be provided in a delivery vehicle or otherwise modified (e.g., in the form of a prodrug), wherein release or activation of the ACE inhibitor is triggered by a physiological stimulus (e.g. pH change, lysosomal enzymes, etc.) Elevated levels in CSF are taken as a good indication of therapeutic efficacy for this class of drugs. Preferably, the pharmaceutical composition of the invention following intranasal adminstration to the mammal yields a peak concentration of the ACE inhibitor in CSF fluid of the mammal that is greater than a nominal therapeutic concentration of the ACE inhibitor in the plasma of the patient. Currently accepted minimal therapeutic concentration (MTC) values in man for rivastigmine and its major active metabolite are on the order of 5 μg/L drug in CSF for 150% (half-maximal) inhibition of acetylcholinesterase activity. For donazepil in rats, MTC was reported as 0.42 nmol/gm. For tacrine in rats, MTC was reported as 3.5 nmol/gm. For TAK-147 (3-[1-(phenylmethyl)-4-piperidinyl]-1-(2,3,4,5-tetrahydro-1H-1-benzazepin-8-yl)-1-propanone) in rats, MTC was reported as 1.1 nmol/gm CSF (Kosasa T et al. 2000. “Inhibitory effect of orally administered donepezil hydrochloride (E2020), a novel treatment for Alzheimer's disease, on cholinesterase activity in rats,” Eur J Pharm 389:173-9; Bobburu J V et al. 2001. “Pharmacokinetic-pharmacodynamic modelling of rivastigmine, a cholinesterase inhibitor, in patients with Alzheimer's disease,” J Clin Pharm 41:1082-90; Cutler N R et al. 1998. “Dose-dependent CSF acetylcholinesterase inhibition by SDZ ENA 713 in Alzheimer's disease,” Acta Neurol Scand 97:244-50; Polinsky R J. 1998. “Clinical pharmacology of rivastigmine,” Clin Ther 20:634-47).

To illustrate the methods and compositions of the invention, the following examples are offered to illustrate exemplary embodiments of the invention. Persons skilled in the art will appreciate that alternate compositions and methods for practicing the invention are within the scope of this disclosure, which is not intended to be limited by the examples below.

### EXAMPLE 1

### Nasal Formulation of Donepezil

| | dry weight |
|---|---|
| Formulation | (grams) |
| Donepezil HCl | 5 |
| α-Cyclodextrin | 5 |
| Benzalkonium Chloride | 0.02 |
| Purified water | q.s. |
| pH = 4.9 | 100 mL |

### EXAMPLE 2

### Nasal Formulation of Donepezil

| | dry weight |
|---|---|
| Formulation | (grams) |
| Donepezil HCl | 5 |
| Polyarginine | 0.2 |
| Benzalkonium Chloride | 0.02 |
| Purified water | q.s. |
| pH = 5.2 | 100 mL |

### EXAMPLE 3

### Nasal Formulation of Donepezil

| | dry weight |
|---|---|
| Formulation | (grams) |
| Donepezil HCl | 5 |
| Benzalkonium Chloride | 0.02 |
| Purified water | q.s. |
| pH = 5.4 | 100 mL |

### EXAMPLE 4

### Nasal Formulation of Donepezil

| | dry weight |
|---|---|
| Formulation | (grams) |
| Donepezil HCl | 5 |
| Chitosan | 0.5 |
| Benzalkonium Chloride | 0.02 |
| Purified water | q.s. |
| pH = 4.06 | 100 mL |

### EXAMPLE 5

### Nasal Formulation of Donepezil

| | dry weight |
|---|---|
| Formulation | (grams) |
| Donepezil HCl | 5 |
| Disodium EDTA | 0.1 |
| Benzalkonium Chloride | 0.02 |
| Purified water | q.s. |
| pH = 4.6 | 100 mL |

### EXAMPLE 6

### Nasal Formulation of Donepezil

| | dry weight |
|---|---|
| Formulation | (grams) |
| Donepezil HCl | 5 |
| Sodium taurocholate | 0.25 |
| Benzalkonium Chloride | 0.02 |
| Purified water | q.s. |
| pH = 5.1 (slightly hazy) | 100 mL |

### EXAMPLE 7

### Nasal Formulation of Donepezil

| | dry weight |
|---|---|
| Formulation | (grams) |
| Donepezil HCl | 5 |
| Sodium taurocholate | 0.3 |
| Benzalkonium Chloride | 0.02 |
| Glycerol | 5.0 |
| Purified water | q.s. |
| pH = 5 | 100 mL |

### EXAMPLE 8

### Nasal Formulation of Donepezil

| | dry weight |
|---|---|
| Formulation | (grams) |
| Donepezil HCl | 5 |
| Sodium taurocholate | 0.3 |
| Benzalkonium Chloride | 0.02 |
| Propylene Glycol | 10.0 |
| Purified water | q.s. |
| pH = 5 | 100 mL |

### EXAMPLE 9

### Bioavailability of Intranasally-Delivered Donepezil in the Rat

A male rat (Rattus norvegicus Sprague-Dawley) weighing about 180 g was prepared surgically with an indwelling venous jugular cannula and lightly anaesthetized during the procedure. The animal was dosed intranasally in the right nostril with an intranasal formulation containing donepezil. Following dosage, the animal's head was raised to prevent the liquid from draining back out of the nasal cavity.

At 5, 10, 15, 30 and 60 min following dosage, paired blood and CSF samples were collected and placed on ice. EDTA was used as an anticoagulant and plasma was separated after centrifugation in a refrigerated centrifuge. All samples were then analyzed for donepezil by HPLC without extraction.

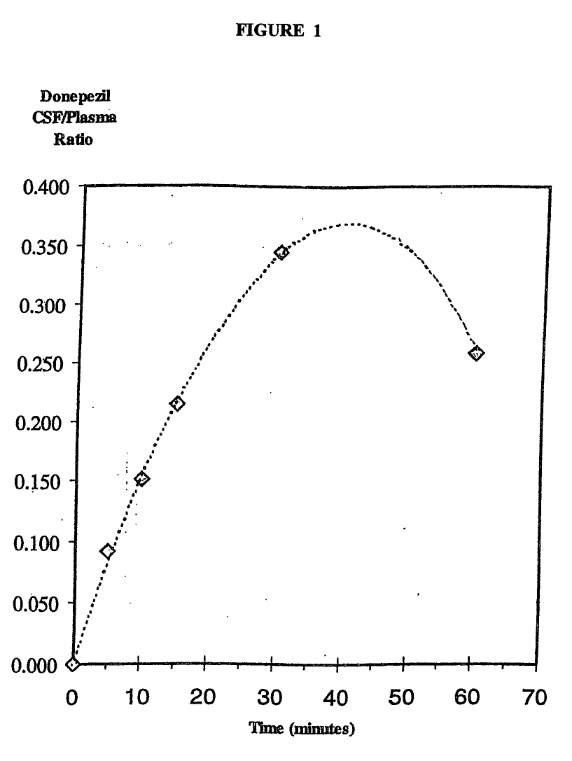

Results were plotted and are shown in FIG. 1. Donepezil is rapidly eliminated in the body by formation of inactive metabolites. However, the sharp changes in CSF to plasma ratio (peak CSF concentration 28.4 nanogram/mL at 30 min), indicates that compartment pools behave independently and that the CSF can be selectively loaded by a route independent and supplementary to plasma loading, possibly involving direct permeation to the subarachnoid plexus. These data are indicative of direct CSF loading by nasal administration according to the compositions and methods of the invention. The CSF concentration achieved is higher than the nominal plasma therapeutic level required for this drug.

### EXAMPLE 10

### Donepezil Dose Tolerance in the Rat

| | dry weight |
|---|---|
| Formulation | (grams) |
| Donepezil HCl | 5 |
| α-cyclodextrin | 12 |
| Purified water | q.s. |
| pH = 4.9 | 100 mL |

A formulation of donepezil 50 mg/mL in α-cyclodextrin 12% was prepared fresh for animal work. Following an approved protocol, two groups of 3 male animals each (Rattus norvegicus Lewis), 150 to 190 gm, were dosed intranasally by instilling 50 μL/kg of the test article or saline control into the right nostril. Care was taken not to damage the nasal mucosa during delivery. Following dosage, the animals head was raised to prevent the liquid from draining back out of the nasal turbinate. The dose was extrapolated from the usual human dose of 5 mg/day and on the basis of the surface area of the nasal cavity in rat versus man (7 cm² for rat versus 80 cm² for human).

Each animal was observed continuously for 60 minutes and thereafter hourly after dosing. A staff veterinarian performed otoscopic examination of the nasal cavity prior to dosing and 4 hours after dosing.

Observations: No signs were noted of nasal irritation such as scratching of the snout, licking or biting of the snout, abnormal posturing, vocalizing, lack of motility, or any sign of pain or distress in the animals dosed with donepezil. Otoscopic examination did not reveal any difference between the saline treated animals and those treated with donepezil, although one treated animal showed slight nasal discharge from both nares immediately after dosing which resolved spontaneously in a few minutes.

Based on the foregoing evidence the formulation was determined to be biocompatible and well tolerated in preclinical testing.

### EXAMPLE 11

### Mucosal Delivery

### Permeation Kinetics and Transmembrane Resistance

The EpiAirway system described herein was developed by MatTek Corp (Ashland, Mass.) as a model of the pseudostratified epithelium lining the respiratory tract. The epithelial cells are grown on porous membrane-bottomed cell culture inserts at an air-liquid interface, which results in differentiation of the cells to a highly polarized morphology. These are normal, human-derived tracheal/bronchial epithelial cells cultured with a proprietary medium to form a pseudostratified, highly differentiated tissue model which closely resembles the epithelial tissue of the respiratory tract. The apical surface is ciliated with a microvillous ultrastructure and the epithelium produces mucus (the presence of mucin has been confirmed by immunoblotting). Tight junctions have been confirmed microscopically and the tissue has a high electrical resistance characteristic of a polarized, impermeable membrane.

Transepithelial resistance for the control tissue typically exceeds 550±125 ohm/cm². The inserts have a diameter of 0.875 cm, providing a surface area of 0.6 cm². The cells are plated onto the inserts at the factory approximately three weeks before shipping. One “kit” consists of 24 units. It has been shown that these differentiated primary cells are functional in paracellular transport of a plurality of drug substances and also in active transport of calcitonin, and provide valuable information predictive of in vivo behavior of nasal formulations. The test is routinely used as a screening tool for formulations and as a means of optimizing paracellular transport.
a. Prior to testing, human respiratory epithelial cells are grown to confluence in a specially designed cup designed for 6 and 24 well cell culture plates. During cell growth and differentiation prior to testing, the cells are exposed to air on the apical side and to medium on the basolateral side. A semipermeable membrane forming the base of the “cup” is used as a cell support. A proprietary medium that is cytokine-free and serum-free is used for cell growth. On arrival, the units are placed onto sterile supports in 6-well microplates. Each well receives 5 mL of proprietary culture medium. The 5 mL volume is just sufficient to provide contact to the bottoms of the units on their stands, but the apical surface of the epithelium is allowed to remain in direct contact with air. The units in their plates are maintained at 37° C. in an incubator in an atmosphere of 5% CO₂ in air for 24 hours. At the end of this time the medium is replaced with fresh medium and the units are returned to the incubator for another 24 hours.
b. In all experiments, the mucosal delivery formulation to be studied is applied to the apical surface of each “cup” containing a confluent monolayer of human respiratory epithelium in a volume of 100 μL. This volume is sufficient to cover the entire apical surface. An appropriate volume of the test formulation at the concentration applied to the apical surface (no more than 100 μL is generally needed) is set aside for subsequent determination of concentration of the drug by HPLC.
c. The units are placed in 6 well plates without stands for the experiment: each well contains 0.9 mL of pre-warmed medium which is sufficient to contact the porous membrane bottom of the unit but does not generate any significant upward hydrostatic pressure on the unit. All cells are routinely held at 37° C. in a humidified CO₂ incubator between manipulations.
d. In order to minimize potential sources of error and avoid any formation of concentration gradients, the units are transferred from one 0.9 mL-containing well to another at each time point in the study. These transfers are made at the following time points, based on a zero time at which the 100 μL volume of test material was applied to the apical surface: 15 minutes, 30 minutes, 60 minutes, and 120 minutes.
e. In between time points the units in their plates are kept in the 37° C. incubator. Plates containing 0.9 mL medium per well are also maintained in the incubator so that minimal change in temperature occurs during the brief periods when the plates are removed and the units are transferred from one well to another using sterile forceps.
f. At the completion of each time point, the medium is removed from the well from which each unit was transferred, and aliquotted into two tubes (one tube receives 700 μL and the other 200 μL) for determination of the concentration of permeated test material.
g. At the end of the 120 minute time point, the units are transferred from the last of the 0.9 mL containing wells to 24-well microplates, containing 0.3 mL medium per well. This volume is again sufficient to contact the bottoms of the units, but not to exert upward hydrostatic pressure on the units. The units are returned to the incubator prior to measurement of transepithelial resistance.
h. In order to minimize errors, all tubes, plates, and wells are prelabeled before initiating an experiment. More details concerning the procedure can be found at the manufacturer's website—www.Mattek.com.
   Protocol for Measuring Transepithelial Electrical Resistance (TEER)
i. Respiratory airway epithelial cells form tight junctions in vitro as well as in vivo, restricting the flow of solutes across the tissue. These junctions confer a transepithelial resistance of several hundred ohms/cm in excised airway tissues. The TEER of control EpiAirway units that have been sham-exposed during the sequence of steps in the permeation study is about 700-800 Ohm/cm². Since permeation of small molecules is proportional to the inverse of the TEER, this value is sufficiently high to provide a major barrier to permeation. The porous membrane-bottomed units without cells, conversely, provide only minimal transmembrane resistance (5-20 Ohm/cm²).
ii. On arrival, the units are placed onto sterile supports in 6-well microplates. Each well receives 5 mL of proprietary culture medium. This DMEM-based medium is serum free but is supplemented with epidermal growth factor and other factors. The medium is tested for endogenous levels of candidate compounds being evaluated for intranasal delivery, and will be free from all such compounds. The 5 mL volume is just sufficient to provide contact to the bottoms of the units on their stands, but the apical surface of the epithelium is allowed to remain in direct contact with air. Sterile tweezers are used in this step and in all subsequent steps involving transfer of units to liquid-containing wells to ensure that no air is trapped between the bottoms of the units and the medium.
iii. The units in their plates are maintained at 37° C. in an incubator in an atmosphere of 5% CO₂ in air for 24 hours. At the end of this time the medium is replaced with fresh medium and the units are returned to the incubator for another 24 hours.
iv. Accurate determinations of TEER require that the electrodes of the ohmmeter be positioned over a significant surface area above and below the membrane, and that the distance of the electrodes from the membrane be reproducibly controlled. The method for TEER determination recommended by MatTek and employed for all experiments here employs an “EVOM”™ epithelial voltohmmeter equipped with a STX2 Electrode pair with internal Ag/AgCl reference electrodes from World Precision Instruments, Inc (Sarasota Fla.; wpiinc.com).
v. The units are read in the following sequence: all sham-treated controls, followed by all formulation-treated samples, followed by a second TEER reading of each of the sham-treated controls.
   Experimental Protocol for Permeation Kinetics
i. A “kit” of 24 EpiAirway units can routinely be employed for evaluating five different formulations, each of which is applied to quadruplicate wells. Each well is employed for determination of permeation kinetics (4 time points), and transepithelial resistance. An additional set of wells is employed as controls, which are sham treated during determination of permeation kinetics, but are otherwise handled identically to the test sample-containing units for determinations of transepithelial resistance. The determinations on the controls are routinely also made on quadruplicate units.
ii. In all experiments, the mucosal delivery formulation to be studied is applied to the apical surface of each unit in a volume of 100 μL, which is sufficient to cover the entire apical surface. An appropriate volume of the test formulation at the concentration applied to the apical surface (no more than 100 μL is generally needed) is set aside for subsequent determination of concentration of the active material by HPLC, ELISA or other designated assay.
iii. The units are placed in 6 well plates without stands for the experiment: each well contains 0.9 mL of medium which is sufficient to contact the porous membrane bottom of the unit but does not generate any significant upward hydrostatic pressure on the unit.
iv. In order to minimize potential sources of error and avoid any formation of concentration gradients, the units are transferred from one 0.9 mL-containing well to another at each time point in the study. These transfers are made at the following time points, based on a zero time at which the 100 μL volume of test material was applied to the apical surface: 15 minutes, 30 minutes, 60 minutes, and 120 minutes.
v. In between time points the units in their plates are kept in the 37° C. incubator. Plates containing 0.9 mL medium per well are also maintained in the incubator so that minimal change in temperature occurs during the brief periods when the plates are removed and the units are transferred from one well to another using sterile forceps.
vi. At the completion of each time point, the medium is removed from the well from which each unit was transferred. These medium permeate samples are kept in the refrigerator if the assays are to be conducted within 24 hours, or the samples are subaliquotted and kept frozen at −80° C. until thawed once for assays. Repeated freeze-thaw cycles are to be avoided.
vii. At the end of the 120 minute time point, the units are transferred from the last of the 0.9 mL containing wells to 24-well microplates, containing 0.3 mL medium per well. This volume is again sufficient to contact the bottoms of the units, but not to exert upward hydrostatic pressure on the units. The units are returned to the incubator prior to measurement of transepithelial resistance.
   Results for Permeability

In vitro data was collected for permeability of donepezil as described in the protocol above. Permeability of donepezil across the tissue layer of the Epi-Airway human respiratory endothelial cell model is reported in Table 1 below as a mass flux in μg/min/cm². As can be seen, donepezil is very mobile in this assay.

TABLE 1
Permeability of Donepezil Formulated for Intranasal Delivery

| | | Flux |
|---|---|---|
| Example | Intranasal Delivery-Enhancing Agent | (μg/min/cm²) |
| 2 | 5% α-cyclodextrin + 0.02% | 7.6 |
| | benzalkonium Cl | |
| 3 | 0.2% polyarginine + 0.02% benzalkonium Cl | 8.9 |
| 4 | 0.02% benzalkonium Cl | 9.4 |
| 5 | 0.5% chitosan + 0.02% benzalkonium Cl | 6.3 |
| 6 | 0.1% disodium EDTA + 0.02% benzalkonium | 9.0 |
| | Cl | |
| 7 | 0.25% sodium taurocholate + 0.02% | 12.4 |
| | benzalkonium Cl | |

Note the increased flux with sodium taurocholate, a well known enhancer acting to increase paracellular transport by opening up and disrupting tight junctions and epithelial membranes.
Results for TEER

In vitro data was collected for tight junction electrical resistance in a respiratory endothelial tissue layer as described in the protocol above and is reported in Table 2 below.

TABLE 2
Effect on TEER of Intranasal Donepezil Formulation

| | | TEER (Ohm/cm²) |
|---|---|---|
| Formula | Intranasal Delivery-Enhancing Agent | as percent control |
| 2 | 5% α-cyclodextrin + 0.02% | 9 |
| | benzalkonium Cl | |
| 3 | 0.2% polyarginine + 0.02% | 8 |
| | benzalkonium Cl | |
| 4 | 0.02% benzalkonium Cl | 6 |
| 5 | 0.5% chitosan + 0.02% benzalkonium Cl | 11 |
| 6 | 0.1% disodium EDTA + 0.02% | 11 |
| | benzalkonium Cl | |
| 7 | 0.25% sodium taurocholate + 0.02% | 9 |
| | benzalkonium Cl | |

Electrical resistance of the sham treated tissue (typically about 500 to 800 Ohm/cm²) was taken as 100%. Care is taken to ensure viability of the cells exposed to each excipient. Decreased transepithelial resistance is indicative of the potency of an excipient in increasing paracellular transport.

### EXAMPLE 12

### Formulation of Rivastigmine for Enhanced Intranasal Mucosal Delivery

Rivastigmine is a recently discovered ACE inhibitor. An exemplary formulation for enhanced mucosal delivery of rivastigmine is prepared as follows:

Rivastigmine Formulation Composition

| Items | % mg/mL |
|---|---|
| Rivastigmine | 2.0 |
| Citric Acid Anhydrous, USP | 6.8 |
| Sodium Citrate Dihydrate, USP | 4.4 |
| 1,3 butanediol, USP | 50.0 |
| Glycerin, USP | 50.0 |
| L-Ascorbic Acid, USP | 0.12 |
| Sodium Metabisulfite, NF | 0.88 |
| Edetate Disodium, USP | 0.2 |
| Benzalkonium Chloride, NF | 0.02 |
| Sodium Hydroxide, NF or Hydrochloric Acid, NF | pH 4 |
| Purified Water, USP (q.s.) | to 100 ml |

The formulation is administered intranasally or by gastric insertion of a capsule containing the commercial formulation to 12 groups of 6 rats prepped with an indwelling jugular cannula and heparin lock. Following an approved protocol, each subject in the experimental groups is given a single dose of intranasal rivastigmine. Subsequently, each subject undergoes lumbar puncture with local anaesthesia (xylocalne s.c.), with the retrieval of 50 uL of cerebralspinal fluid (CSF). A paired blood sample from each animal is collected. By assigning groups to different timepoints, the whole PK curve can be assembled. The first CSF sample is collected 5 minutes post dosing and subsequent samples were collected at 20, 50, 75, 100 and 400 minutes. The procedure is repeated using oral rivastigmine for the control groups.

CSF samples are frozen until analysis. The data shows that rivastigmine in plasma follows a PK that is independent of the kinetics in CSF. Furthermore, when administered intranasally, the rivastigmine concentration in CSF peaks at a level higher than therapeutic levels reported for plasma following oral dosage in man. The plasma curve (AUC) for intranasal rivastigmine is shown to be remarkable by comparison to the plasma AUC for rivastigmine administered orally. We attribute this to the effects of first pass clearance on reducing AUC for drugs administered orally.

Optionally, treated animals may be tested for memory enhancement in a water maze learning model or other model of cognitive functioning.

### EXAMPLE 13

### Formulation of Huperzine A (Selegine) for Enhanced Intranasal Mucosal Delivery

Huperzine A is a plant derived, naturally occurring ACE inhibitor and is available from Sigma Chemicals (St Louis Mo.). An exemplary formulation of Huperzine A for enhanced mucosal delivery of huperzine as follows:

Huperzine Formulation Composition

| Items | % mg/mL |
|---|---|
| Huperzine A | 5.0 |
| Citric Acid Anhydrous, USP | 6.8 |
| Sodium Citrate Dihydrate, USP | 4.4 |
| Propylene Glycol, USP | 70.0 |
| Glycerin, USP | 50.0 |
| L-Ascorbic Acid, USP | 0.12 |
| Edetate Disodium, USP | 0.2 |
| Benzalkonium Chloride, NF | 0.2 |
| Sodium Hydroxide, NF or Hydrochloric Acid, NF | pH 3.5 |
| Purified Water, USP (qs) | to 100 ml |

Optionally, treated animals may be tested for memory enhancement in a water maze learning model or other model of cognitive functioning.

### EXAMPLE 14

### Production and Use of Carboxylate Salts of Galantamine as ACE Inhibitors for Mucosal Delivery to Treat and Prevent CNS Disorders in Mammals

An important ACE inhibitor for the prevention and treatment of diseases and disorders of the CNS within the invention is galantamine. Currently galantamine is delivered orally as the hydrobromide salt in tablet form or oral solution. However, as an orally administered drug, galantamine reaches a maximum inhibition of acetylcholinesterase one hour after administration. It is possible that an intranasal formulation could result in a maximum inhibition of acetylcholinesterase in a shorter amount of time than the orally administered galantamine. However, to intranasally deliver therapeutically relevant doses of galantamine, the concentration of drug would have to be in excess of 40 mg/mL, for example, preferably 80 mg/mL. This is dictated by the volume limitation for nasal spray dosing (˜100 μL per nostril per spray). However, the solubility of the currently available form, namely galantamine hydrobromide, does not achieve this goal. Thus, there is a need to produce formulations of galantamine that have increased solubility.

The present invention fills this need by providing for novel galantamine carboxylate salts such as galantamine gluconate, galantamine lactate, galantamine glucarate and galantamine citrate. It has been unexpectedly discovered that the novel galantamine carboxylate salts of the present invention are substantially more soluble than galantamine hydrobromide.

In exemplary embodiments of the invention, carboxylate salts of galantamine are produced by replacing the bromide of galantamine hydrobromide with a carboxylate anion which (1) provides higher solubility compared to bromide and (2) is a weaker anion than bromide (as illustrated by behavior upon ion exchange, described below). Examples of appropriate counter anions are carboxylates of the form:
R—(COO⁻)ₓ
where x≧1 and R is an alkyl group. In one embodiment, R contains one or more hydroxyl groups on the carbon backbone. Examples of such specific embodiments include, but are not limited to, gluconate, lactate, glucarate, benzoate, acetate, salicylate, tartrate, mesylate, tosylate, maleate, fumarate, stearate and citrate.

In related embodiments, the present invention provides methods for producing a galantamine carboxylate salt in which a solution of a carboxylate salt formed producing carboxylate anions in solution. This solution containing the carboxylate anions is applied to an anion exchange resin under conditions wherein the carboxylate anions bind to the anion exchange resin. Galantamine hydrobromide is dissolved in an appropriate solvent such as water under conditions where bromide ions are formed in solution. The galantamine hydrobromide solution is then added to the anion exchange resin under conditions wherein the carboxylate anions are displaced and the bromide anions bind to the anion exchange resin resulting in the formation of a galantamine carboxylate salt or complex. Common types of anion exchange resins are diethylaminoethyl (DEAE-) and quaternary amino ethyl- (TEAE-, QAE) substituents attached directly hydroxyl groups on the matrix of the resin. Suitable ion exchange processes include, but are not limited to, batch processes using a resin slurry, and also a process using a resin packed in a column.

The current invention encompasses salt forms of galantamine with increased solubility compared to galantamine hydrobromide and methods for their generation. Said generation can be accomplished, for example, by salt exchange on an anion exchange resin, generally used for purification of proteins and peptides. Taking advantage of its strong-anion binding capability, a quaternary ammonium anion exchange resin is first saturated with R—(COO⁻)ₓ. After this weak anion is bound to the resin, galantamine hydrobromide is loaded on the resin. The bromide, being a stronger anion, displaces R—(COO⁻)ₓ on the resin and the galantamine elutes with a new, and more soluble, salt form. Elemental analysis confirmed 900 fold depletion of bromide in the eluted fractions from the resin. Water can then be removed to concentrate the new galantamine salt.

The present invention facilitates the development of nasal formulations by removing a previously existing barrier of concentration limitations. With the new salt forms, solubility can be increased at least ten fold over the concentration of galantamine hydrobromide. The maximum concentration of galantamine hydrobromide in water is about 35 mg/mL (121 mM). The generally reported solubility of galantamine hydrobromide in water is 50 mM. Surprisingly the novel galantamine carboxylate salts galantamine gluconate and galantamine lactate both have solubilities in water of approximately 400 mg/mL (1.39 M). Typical yields at the lab scale for the current ion exchange batch process are 89-97%. The examples below provide additional details of the methodology and the experimental data.

A batch process is a process in which the feed is charged into the system at the beginning of the process, and the products are removed all at once some time later. No mass crosses the system boundaries between the time the feed is charged and the time the product is removed.

In a continuous process inputs and outputs flow continuously throughout the duration of the process.

With the concentration barrier removed, formulation of galantamine for nasal delivery can now be achieved without the addition of excipients (e.g., solubility enhancers) to address solubility concerns. The small volume necessary for nasal delivery is no longer a limiting factor for galantamine intranasal formulation development. Even the highest dose generally delivered orally, 24 mg galantamine, can feasibly be delivered in a single nasal spray dose of 100 μL.

These salts of galantamine typically provide at least a two-fold increase, often at least a five-six fold increase, and up to a 10-fold, 15-fold or even 20-fold or greater increase in solubility compared to galantamine hydrobromide. These galantamine salts can be administered to an individual to inhibit acetylcholinesterase in the treatment of such diseases as Alzheimer's disease, atony of the smooth muscle of the intestinal tract and urinary bladder, glaucoma, myasthenia gravis, and termination of the effects of competitive neuromuscular blocking drugs. An exemplary dosage of these carboxylate salts of galantamine is between about 16-32 mg given twice a day. Other, lower or higher, doses and dosing schedules as contemplated herein will also be useful, depending on patient and treatment criteria as noted above.

### EXAMPLE 15

### Galantamine Salt Exchange: Bromide to Gluconate Using QAE SEPHADEX® Slurry in a Batch Ion-Exchange Process

Galantamine gluconate was produced according to the following procedure.

Study Design

| Sample | Composition | Comments | Testing |
|---|---|---|---|
| 1 (100 mg/4 mL) | 25 mg/mL Galantamine HBr | | pH |
| | | | HPLC |
| | | | Elemental |
| | | | Analysis |

Materials

| | | | F.W. |
|---|---|---|---|
| Reagent | Grade | Vendor | Data |
| Galantamine HBr | | Tocris Cookson | 377.28 |
| Purified Water | | | |
| QAE SEPHADEX ® | | Pharmacia | |
| Sodium Gluconate | USP | Spectrum | 218.14 |

QAE SEPHADEX® Preparation

QAE SEPHADEX® has a meq/g of 3.0+/−0.4. To be sure that the anion exchange sites were in a 100 fold excess of galantamine, 8.88 g dry powder QAE Sephadex was pre-swollen in water for 2 days at room temperature in a 250 mL beaker. (See the following chart for calculations to determine amount of QAE SEPHADE® required.)

| Galantamine HBr | | | QAE SEPHADEX ® | | | |
|---|---|---|---|---|---|---|
| total mg | MW | moles | fold excess | eq | eq/g | g |
| 100 | 377.28 | 0.000265 | 100 | 0.026506 | 0.003 | 8.835 |
| 50 | 377.28 | 0.000133 | 100 | 0.013253 | 0.003 | 4.418 |

After QAE SEPHADEX® was swollen, it was rinsed with 1M Sodium Gluconate, pH 5.0 three times to fully bind gluconate to all the anion binding sites. The slurry was then washed three times with purified water to remove excess salt in solution.

Galantamine Sample Preparation

A 25 mg/mL Galantamine HBr solution was prepared by adding 100 mg galantamine to 4 mL purified water. Solution was vortexed to dissolve galantamine.

Ion Exchange

After QAE Sephadex resin had been prepared, the galantamine HBr solution was added to the batch resulting in bromide ions binding to the QAE SEPHADE® in the slurry and gluconate eluting off the resin and becoming complexed with the galantamine. The solution was left in the slurry for 30 min, with mild agitation at room temperature. The galantamine gluconate was recovered from the resin by filtration. Samples were centrifuged to clear any particles from the resin that are in solution.

Removing Water

Samples were lyophilized using the BenchTop 2K lyophilizer from Virtis (Gardner, N.Y.). Samples were dried in 50 mL centrifuge tubes to maximize surface area space.

Solubility Test

Dried galantamine was weighed in 50 mL. A minimum volume of purified water was be added to each sample slowly to maximize concentration of galantamine in solution. After Galantamine was dissolved in water, the solution was removed from the 50 mL tube and the tube was weighed again to determine the amount of galantamine in the tube by weight loss. The final concentration was determined by HPLC.

HPLC Methods

All samples (and corresponding “placebos”) were diluted 1:150 with 50 mM ammonium formate. Samples were assayed using an isocratic LC (Waters Alliance) method with UV detection.

| | |
|---|---|
| Column: | Waters Symmetry Shield, C18, 5 μm, |
| | 25 × 0.46 cm |
| Mobile phase: | 1.5% ACN in 50 mM ammonium formate, pH 3.0 |
| Flow rate: | 1.3 mL/min |
| Column temperature: | 30° C. |
| Calibration curve: | 0-400 μg/mL Galantamine HBr (Tocris) |
| Detection: | UV at 285 nm |

Results:

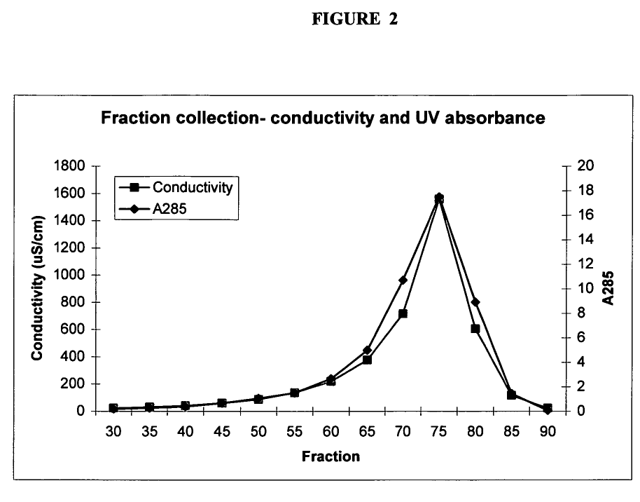

Using the above-described procedure resulted in a 98.23% recovery of galantamine gluconate. FIG. 2 illustrates the UV absorbance of the galantamine fractions obtained. The solubility of galantamine gluconate produced according to the inveniotn was at least 238 mg/mL, which represents approximately a 5.75 fold increase in solubility over the solubility of galantamine hydrobromide. Elemental analysis confirmed a 263-fold reduction in the ratio of bromide to galantamine, confirming that the bromide salt was successfully exchanged.

### EXAMPLE 16

### Galantamine Salt Exchange: Bromide to Lactate Using QAE SEPHADEX® Slurry in a Batch Ion-Exchange Process

Study Design

| Sample | Composition | Testing |
|---|---|---|
| 1 (100 mg/4 mL) | 25 mg/mL Galantamine HBr | pH |
| | | HPLC |
| | | Elemental |
| | | Analysis |

Materials

| Reagent | Grade | Vendor | F.W. |
|---|---|---|---|
| Galantamine HBr | | Tocris Cookson | 377.28 |
| Purified Water | | | |
| QAE SEPHADEX ® | | Pharmacia | |
| Sodium Lactate Solution, 60% | USP | Spectrum | 112.06 |

Galantamine lactate was produced according to the same procedure that galantamine gluconate was produced except that sodium lactate was the carboxylate salt instead of sodium gluconate.

Results:

The process described above produced an 89.74% yield of galantamine lactate. The solubility of the galantamine lactate was about 314 mg/mL, which was more than about a 9-fold increase in solubility over galantamine hydrobromide. Elemental analysis confirmed a 227-fold reduction in the ratio of bromide to galantamine, confirming that the bromide salt was successfully exchanged.

### EXAMPLE 17

### Exchanging Salt Forms of Galantamine-Gluconate for Bromide and Lactate for Bromide

Study Design

| Sample | Composition | Comments | Testing |
|---|---|---|---|
| 1 (200 mg/8 mL) | 25 mg/mL Galantamine | Gluconate salt | pH |
| | HBr | exchange | HPLC |
| | | | EA |
| 1 (200 mg/8 mL) | 25 mg/mL Galantamine | Lactate salt | pH |
| | HBr | exchange | HPLC |
| | | | EA |

Materials

| | | | F.W. |
|---|---|---|---|
| Reagent | Grade | Vendor | Data |
| Galantamine HBr | | Tocris Cookson | 377.28 |
| Purified Water | | | |
| QAE sephadex | | Pharmacia | |
| Sodium Gluconate | USP | Spectrum | 218.14 |
| Sodium Lactate Solution, 60% | USP | Spectrum | 112.06 |

QAE SEPHADEX® Preparation

QAE SEPHADEX® has a meq/g of 3.0+/−0.4. To be sure that the anion exchange sites were in a 100 fold excess of galantamine, 2 separate aliquots of 17.6 g dry powder QAE SEPHADEX® were pre-swollen in water for 2 days at room temperature. (See the following chart for calculations to determine amount of QAE SEPHADEX® required.)

| Galantamine HBr | | | QAE sephadex | | | |
|---|---|---|---|---|---|---|
| total mg | MW | moles | fold excess | eq | eq/g | g |
| 100 | 377.28 | 0.000265 | 100 | 0.026506 | 0.003 | 8.835 |
| 200 | 377.28 | 0.000530 | 100 | 0.053011 | 0.003 | 17.67 |

After the QAE SEPHADEX® was swollen, it was rinsed with either 1M sodium gluconate three times or 1 M Sodium Lactate four times to fully bind gluconate or lactate to all the anion binding sites. The slurry was then washed three times with purified water to remove excess salt in solution.

Galantamine Sample Preparation

Two 25 mg/mL Galantamine HBr solutions were prepared by adding 200 mg galantamine to 8 mL purified water. The solutions were vortexed to dissolve the galantamine.

Ion Exchange

After the QAE SEPHADEX® resin was prepared, the Galantamine HBr solution was added in batch. Bromide ion bound to QAE SEPHADEX® and gluconate or lactate complexed with galantamine. The solution was left on the beads for 60 min, and mildly agitated at room temperature. The galantamine gluconate or galantamine lactate were recovered from the resin by filtration. Multiple fractions were collected from the resin by adding water to the resin after the initial sample was collected. This is to maximize galantamine recovery. Samples will be centrifuged to clear any particles from the resin that are in the recovered fractions. Concentration was determined by HPLC.

Removing Water

Samples were lyophilized using the BenchTop 2K lyophilizer from Virtis (Gardner, N.Y. model # 393775). Samples were dried in 50 mL centrifuge tubes to maximize surface area space.

Solubility Test

Dried galantamine in 50 mL tubes will be weighed. A minimum volume of purified water will be added to each sample slowly to maximize concentration of galantamine in solution. After Galantamine has dissolved in water, the solution will be removed from the 50 mL tube and the tube will be weighed again to determine the amount of galantamine in the tube by weight loss.

HPLC Methods

All samples (and corresponding “placebos”) diluted 1:150 with 50 mM ammonium formate, pH 3.0, i.e. 10 μL sample mixed with 1490 μL diluent. Samples assayed using an isocratic LC (Waters Alliance) method with UV detection.

| | |
|---|---|
| Column: | Waters Symmetry Shield, C18, 5 um, 25 × 0.46 |
| | cm |
| Mobile phase: | 1.5% ACN in 50 mM ammonium formate, pH 3.0 |
| Flow rate: | 1.3 ml/min |
| Column temperature: | 30° C. |
| Calibration curve: | 0-400 μg/mL Galantamine HBr (Tocris) |
| Detection: | UV at 285 nm |

Results:

The process described above produced an 83% yield of galantamine lactate. The solubility of the galantamine lactate was at least 395 mg/mL, which was more than an 1′-fold increase in solubility over galantamine hydrobromide. Furthermore, the process above-described process produced an 87% yield of galantamine gluconate. The solubility of the galantamine gluconate was at least 395 mg/mL, which was more than an 1′-fold increase in solubility over galantamine hydrobromide.

### EXAMPLE 18

### Galantamine Salt Exchange: Bromide to Lactate Using a 1 mL QSEPHAROSE® Column

Study Design

| Sample | Composition | Comments | Testing |
|---|---|---|---|
| 1 (8 mg/266.7 | 30 mg/mL | Lactate salt exchange | UV(285 nm) |
| μL) | Galantamine | 10 fold excess of resin | osm |
| | HBr | | conductivity |
| | | | Br— ion |
| | | | HPLC |
| 2 (4 mg/133.3 | 30 mg/mL | Lactate salt exchange 20- | UV(285 nm) |
| μL) | Galantamine | fold excess of resin | osm |
| | HBr | | conductivity |
| | | | Br— ion |
| | | | HPLC |
| 3 (1.6 mg/53.3 | 30 mg/mL | Lactate salt exchange | UV(285 nm) |
| μL) | Galantamine | 50 fold excess of resin | osm |
| | HBr | | conductivity |
| | | | Br— ion |
| | | | HPLC |
| 4 (0.8 mg/26.7 | 30 mg/mL | Lactate salt exchange 100- | UV(285 nm) |
| μL) | Galantamine | fold excess of resin | osm |
| | HBr | | conductivity |
| | | | Br— ion |
| | | | HPLC |

Materials

| Reagent | Grade | Vendor | F.W. |
|---|---|---|---|
| Galantamine HBr | | Tocris Cookson | 377.28 |
| Purified Water | | | |
| HiTrap Q SEPHAROSE ® | | Amersham Biosciences | |
| FF | | | |
| Sodium Lactate Solution, | USP | Spectrum | 112.06 |
| 60% | | | |

HiTrap Q SEPHAROSE® FF column preparation

HiTrap Q SEPHAROSE® FF columns were equilibrated following the instructions manual. First, a 1 mL column was washed with 5 column volumes of water to remove preservatives and storage buffer. The column was subsequently washed with 5 column volumes of 1 M sodium lactate to prime the column. Finally, the column was washed with 5-10 column volumes of water to remove the excess salt. Eluent was monitored with a conductivity meter to assess that all excess salt was no longer eluting from column.

Galantamine Sample Preparation

1 mL of 30 mg/mL Galantamine HBr solution was prepared. The solutions were vortexed to dissolve galantamine.

To determine the minimal amount of excess resin that is required to successfully exchange galantamine HBr for galantamine lactate, varying amounts of galantamine were loaded on the 1 mL columns to test 50×, 20×, and 10× excess of the ionic capacity of Q SEPHAROSE® to the moles of galantamine present. The ionic capacity of the resin is 0.18-0.25 mmole/mL gel. (See the following chart for calculations to determine amount of galantamine to load on a 1 mL column.)

| Galantamine | | | | | |
|---|---|---|---|---|---|
| HBr | | excess resin | | Q SEPHAROSE ® | |
| mg | mmoles | fold | mmole | mL (low capacity) | mL (high capacity) |
| 1.6 | 0.004 | 50 | 0.212 | 1.18 | 0.85 |
| 8.0 | 0.021 | 10 | 0.212 | 1.18 | 0.85 |

Ion Exchange

After the HiTrap Q SEPHAROSE® column was prepared, the Galantamine HBr solution was loaded with a syringe at approximately 1 mL/min. Bromide ion bound to Q SEPHAROSE® and lactate complexed with galantamine. The galantamine lactate was be eluted from the column by washing the column with 5-10 column volumes of water. Multiple 1 mL fractions were collected from the column to maximize galantamine recovery. Samples were tested for conductivity, osmolarity, pH, and for galantamine content by measuring A₂₈₅. Concentration was determined by HPLC.

Removing Water

Samples were lyophilized using the BenchTop 2K lyophilizer from Virtis (Gardner, N.Y.). 6 Samples (2-4 mL total vol) were dried in 15 mL centrifuge tubes to maximize surface area space.

Solubility Test

A minimum volume of purified water was added to each sample slowly to maximize concentration of galantamine in solution. After Galantamine was dissolved in water, the solution was transferred to a microcentrifuge tube.

Measurement of Osmolarity

Samples were measured with an Advanced Micro Osmometer, Model 3300, S/N 9812146H from Advanced Instruments Inc. (Norwood, Mass.) using a 20 microliter Sampler, and disposable sample tips.

Conductivity Measurements

Conductivity was measured using the Traceable® Portable Conductivity Meter, with probe from VWR International.

Bromide Ion Concentration Determination

Bromide ions were measured using an Ionplus Sure Flow Bromide probe, Orion model 9635BN with Orion 520Aplus pH meter, Thermo Electron Corp (USA).

UV Spectrophotometer Measurements

UV absorbance were read on a μQuant optical density plate reader, by Biotek Instruments (Winooski, Vt.) at 285 nm using KCJr software. 100 μL of sample were loaded in each well. Water was used as a blank. To get an estimate of galantamine concentration, three controls were loaded: 0.333 mg/mL, 0.111 mg/mL and 0.055 mg/mL Galantamine HBr in water. From these, a line was plotted and the concentrations of the fractions from the columns were determined.

HPLC Methods

Samples were assayed using a gradient LC (Waters Alliance) method with UV detection.

| | |
|---|---|
| Column: | Waters Symmetry Shield, C18, 5 um, 25 × 0.46 |
| | cm |
| Mobile phase: | A: 1.5% ACN in 50 mM ammonium formate, |
| | pH 3.0 B: ACN |
| Flow rate: | 1.3 mL/min |
| Column temperature: | 30° C. |
| Calibration curve: | 0-400 μg/mL Galantamine HBr (Tocris) |
| Detection: | UV at 285 nm |
| Sample diluent: | Buffer A |

Results:

The process described above produced a 91% yield of galantamine lactate. The solubility of the galantamine lactate was at least 217 mg/mL, which was more than a 6-fold increase in solubility over galantamine hydrobromide. Detection of bromide ions using the bromide ion specific probe demonstrated about a 240-fold reduction in the ratio of bromide to galantamine, confirming that the bromide salt was successfully exchanged.

### EXAMPLE 19

### Galantamine Salt Exchange: Bromide to Gluconate Using a 1 ml Q SEPHAROSE® Column

Study Design

| Sample | Composition | Comments | Testing |
|---|---|---|---|
| 2 (4 mg/133.3 | 30 mg/mL | Galantamine HBr solution | UV(285 nm) |
| μL) | Galantamine | made for Gal-022 | conductivity |
| | HBr | Gluconate salt exchange | Br— ion |
| | | 20 fold excess of resin | HPLC |

Materials

| Reagent | Grade | Vendor | F.W. |
|---|---|---|---|
| Galantamine HBr | | Tocris Cookson | 377.28 |
| Purified Water | | | |
| HiTrap Q SEPHAROSE ® | | Amersham Biosciences | |
| FF | | | |
| Sodium Gluconate | USP | Spectrum | 218.14 |

Galantamine gluconate was produced according to the procedures above except that sodium gluconate was the carboxylate salt instead of sodium lactate.

Results:

The process described above produced a 99% yield of galantamine gluconate. The solubility of the galantamine gluconate was at least 215 mg/mL, which was more than a 6-fold increase in solubility over galantamine hydrobromide. Detection of bromide ions using the bromide ion specific probe demonstrated about a 228-fold reduction in the ratio of bromide to galantamine, confirming that the bromide salt was successfully exchanged.

### EXAMPLE 20

### Galantamine Salt Exchange: Bromide to Lactate on a 1 L Q SEPHAROSE® column

Study Design

| Sample | Composition | Comments | Testing |
|---|---|---|---|
| 1 (1 g/33.3 mL) | 30 mg/mL | Lactate salt exchange | UV(285 nm) |
| | Galantamine | 100 fold excess of resin | osm |
| | HBr | | conductivity |
| | | | Br— ion |
| | | | HPLC |

Materials

| Reagent | Grade | Vendor | F.W. |
|---|---|---|---|
| Galantamine HBr | | Tocris Cookson | 377.28 |
| Purified Water | | | |
| Q SEPHAROSE ® FF | | Amersham | |
| | | Biosciences | |
| Sodium Lactate Solution, 60% | USP | Spectrum | 112.06 |

Q SEPHAROSE® FF Biosciences

Sodium Lactate Solution, 60% USP Spectrum 112.06 Q SEPHAROSE® FF column packing A column was first packed in an XK50/60 column body from Amersham Biosciences with Q SEPHAROSE® Fast Flow resin, according the instructions from Amersham. Briefly, the 20% Ethanol solution was decanted from the Q SEPHAROSE® resin and a slurry was prepared that contains roughly 75% resin and 25% water. The resin was then degassed under a vacuum. The Column was prepared by flushing the bottom with water to purge the system of air. The column was packed with the addition of a RK50 reservoir from Amersham. The degassed resin was poured in one smooth motion down the length of the column along a side wall. The column was attached to a BioRad Econo Pump peristaltic pump (s/n 700 BR 09961). The upper limit for a linear flow rate for the resin, as quoted in the instructions, is 400-700 cm/hr. The maximum flow rate for this pump is 20 mL/min.

Column Pre-Washes

Once the column bed was packed and a constant bed height reached, the adaptor was attached and the column was washed with 3-5 column volumes of water. The eluant was monitored by conductivity to confirm that the column reached equilibrium.

After the first wash with water, the column was washed with 1 M Sodium Lactate for 5 column volumes or until the conductivity of the eluant ceased to change and matched that of the solution being loaded on the column. The 1 M sodium lactate was degassed before use. The flow rate was 12 mL/min.

After the 1 M salt wash, the column underwent a second water wash to remove excess salt from the column. The water was degassed before use. The eluant was monitored by conductivity and this step continued until either 10 column volumes of water were used or the conductivity dropped below 30 μS/cm. The flow rate was 12 mL/min.

Galantamine Sample Preparation

33.3 mL of a 30 mg/mL Galantamine HBr solution was prepared. The solution was vortexed to dissolve the galantamine.

Ion Exchange

After the Q SEPHAROSE® column was prepared, the Galantamine HBr solution was loaded. Bromide ion bound to the Q SEPHAROSE® and lactate complexed with the galantamine. The galantamine lactate was eluted from the column by washing the column with 2-5 column volumes of water. 7.5 mL fractions were collected from the column to maximize galantamine recovery and separation from excess salt. Samples were tested for conductivity, osmolarity, and for galantamine content by measuring A₂₈₅. Concentration were determined by HPLC.

Removing Water

Samples were lyophilized using the BenchTop 2K lyophilizer from Virtis (Gardner, N.Y. model # 393775. Samples (15-20 mL total vol) were dried in 40 mL glass vials to maximize surface area space.

Solubility Test

A minimum volume of purified water was added to each sample slowly to maximize concentration of galantamine in solution. After galantamine was dissolved in water, the solution was transferred to a microcentrifuge tube.

Measurement of Osmolarity

Samples were measured with an Advanced Micro Osmometer, Model 3300, from Advanced Instruments Inc. (Norwood, Mass.) using a 20 microliter Sampler, and disposable sample tips.

Conductivity Measurements

Conductivity was measured using the Traceable® Portable Conductivity Meter, with probe from VWR International.

Bromide Ion Concentration Determination

Bromide ions were measured using an Ionplus Sure Flow Bromide probe, Orion model 9635BN with Orion 520Aplus pH meter, Thermo Electron Corp (USA).

UV Spectrophotometer Measurements

UV absorbance was read on a μQuant optical density plate reader, by Biotek Instruments (Winooski, Vt.) at 285 nm using KCJr software. 100 μL of sample will loaded in each well. Water was used as a blank. To get an estimate of galantamine concentration, three controls were loaded: 0.333 mg/mL, 0.111 mg/mL and 0.0370 mg/mL Galantamine HBr in water. From these, a line was plotted and the concentrations of the fractions from the columns were determined.

HPLC Methods

Samples were assayed using a gradient LC (Waters Alliance) method with UV detection.

| | |
|---|---|
| Column: | Waters Symmetry Shield, C18, 5 um, 25 × 0.46 |
| | cm |
| Mobile phase: | A: 1.5% ACN in 50 mM ammonium formate, |
| | pH 3.0 B: ACN |
| Flow rate: | 1.3 ml/min |
| Column temperature: | 30° C. |
| Calibration curve: | 0-400 μg/mL Galantamine HBr (Tocris) |
| Detection: | UV at 285 nm |
| Sample diluent: | Buffer A |

Galantamine Recovery from Column

Every 5^{th} fraction was monitored by conductivity and by UV absorbance at 285 nm. Results are shown in Table 3 below (766.8 mg Galantamine (1.000 mg Galantamine HBr) was loaded on the column).

TABLE 3
Galantamine Recovery

| | | | | Total | | | Bromide |
|---|---|---|---|---|---|---|---|
| | | volume | Galantamine | Galantamine | Recovery | Bromide | remaining |
| Pool | Fractions | (mL) | (mM) | (mg) | % | (mM) | % |
| A | 30-44 | 109 | 0.5 | 15.0 | 2.0% | 0.0013 | 0.27% |
| B | 45-59 | 109 | 1.9 | 59.5 | 7.8% | 0.0026 | 0.14% |
| C | 60-69 | 71 | 7.1 | 144.9 | 18.9% | 0.0084 | 0.12% |
| D | 70-80 | 79 | 22.1 | 501.7 | 65.4% | 0.0244 | 0.11% |
| E | 83-90 | 57 | 1.4 | 22.9 | 3.0% | 0.0030 | 0.22% |
| Total (mg): | | | | 744.0 | 97.0% | | |

Galantamine concentration determined by HPLC
Bromide concentration determined by bromide ion probe

### EXAMPLE 21

### Permeation Enhancement by PN159 For Galantamine

The present example demonstrates efficacy of an exemplary peptide of the invention, PN159 to enhance epithelial permeation for a small molecule ACE inhibitor, galantamine. In this example, a combination of one or more permeabilizing peptides with galantamine is described. Useful formulations in this context can include a combination of a small molecule ACE inhibitor such as galantamine with a permeabilizing peptide alone, or with one or more additional delivery or permeation enhancers. These formulations may also contain buffers, tonicifying agents, pH adjustment agents, stabilizers and/or preservatives.

As noted above, galantamine is an ACE inhibitor and an allosteric agonist of nicotinic acetylcholine receptor. By virtue of these activities, galantamine is useful within the invention for the prevention and treatment of diseases and disorders of the CNS. Diseases amenable for treatment according to methods and compositions of the invention employing galantamine as an ACE inhibitory include, inter alia, neurological conditions associated with memory loss, cognitive impairment and dementia in mammals, including Alzheimer's Disease, Parkinson's-type dementia, certain forms of schizophrenia, forms of delirium, and dementia. Thereapeutic efficacy of treatment methods and compositions in this context can be demonstrated by showing reduction in the incidence, severity, or recurrence of one or more symptoms of a CNS disorder in a test subject treated with a galantamine formulation as described herein as compared to a suitable placebo-treated or other control subject. Typically, a formulation or method of the invention will yield at least a 10% reduction in the incidence, severity, or recurrence of one or more symptoms correlated with the targeted CNS disease. More often, the formulations and methods of the invention yield at least a 15-25%, 30-50%, 50-75%, or 75-90% reduction in the incidence, severity, or recurrence of one or more symptoms correlated with the targeted CNS disease. In certain embodiments, the formulations and methods of the invention will yield as much as a 90-95% or greater reduction in the incidence, severity, or recurrence of one or more symptoms correlated with the targeted CNS disease, and in some cases may yield total prevention, elimination, or remission of one or more, and sometimes all, disease symptoms. Pathological symptoms alleviated or prevented in Alzheimer's disease sibjects, for example, may include degeneration of cholinergic neurons in the subcortical regions and of neuronal pathways that project from the basal forebrain, and associated cognitive impairment (which may be meausured by various known cognitive performance assessment tools for evaluating memory, attention, learning, and other cognitive processes).

Additional description pertaining to intranasal delivery of ACE inhibitors, including galantamine, within the instant invention is provided, for example, in U.S. patent application Ser. No. 10/439,108, filed by Quay on May 15, 2003. In addition, supplemental description herein regarding salt exchange of galantamine to yield carboxylate salt forms to significantly increase drug solubility for use within the invention is provided, for example, in U.S. patent application Ser. No. 10/831,031, filed by Quay et al., on Apr. 23, 2004.

The present example demonstrates that combinatorial formulations and coordinate administration methods combining galantamine with a permeabilizing peptide, exemplified by PN159, yield enhanced permeation of galantamine across the nasal mucosa. This increase in drug permeation is unexpected because galantamine is a small molecule that can permeate the nasal epithial membrane independently. The significant enhancement of galantamine permeation across epithelia mediated by addition of permeabilizing peptides that enhance permeation of larger molecules, such as therapeutic PTH and peptide YY peptides, is therefore surprising. In particulare, such permeabilizing peptides would not ordinarily be expected to significantly increase permeation of small molecule drugs such as galantamine across epithelial tissue layers. The invention therefore will facilitate nasal delivery of galantamine and other small molecule drugs by increasing their bioavailability.

In the present studies, 40 mg/ml galantamine in the lactate salt form was combined with 25, 50, and 100 μM PN159 in solution, pH 5.0 and osmolarity 270 mOsm. The combination was tested using an in vitro epithial tissue model to monitor galantamine permeation, transepithelial electrical resistance (TER), and the cytotoxicity of the formulation by LDH and MTT assays as described above. Permeation measurements for galantamine were conducted by standard HPLC analysis, as follows.

HPLC Analysis

Galantamine concentration in the formulation and in the basolateral media (permeation samples) was determined using an isocratic LC (Waters Alliance) method with UV detection.

| | |
|---|---|
| Column: | Waters Symmetry Shield, C18, 5 um, 25 × 0.46 |
| | cm |
| Mobile phase: | 5% ACN in 50 mM ammonium formate, pH 3.0 |
| Flow rate: | 1 ml/min |
| Column temperature: | 30° C. |
| Calibration curve: | 0-400 μg/ml Galantamine HBr |
| Detection: | UV at 285 nm |

Based on the foregoing studies, the utility of the permeabilizing peptides of the invention, exemplified by PN159, for improving transmucosal delivery of small molecules was demonstrated. Galantamine was chosen as a model low molecular weight drug, and the results for this molecule are considered predictive of permeabilizing peptide activity for other small molecule drugs. To evaluate permeabilizing activity in this context, 40 mg/ml galantamine in the lactate salt form was combined with 25, 50, and 100 μM PN159 in solution, pH 5.0 and osmolarity ˜270 mOsm. The combination was tested using an in vitro epithelal tissue model to monitor galantamine permeation, transepithelial electrical resistance (TER), and the cytotoxicity of the formulation by LDH and MTT assays.

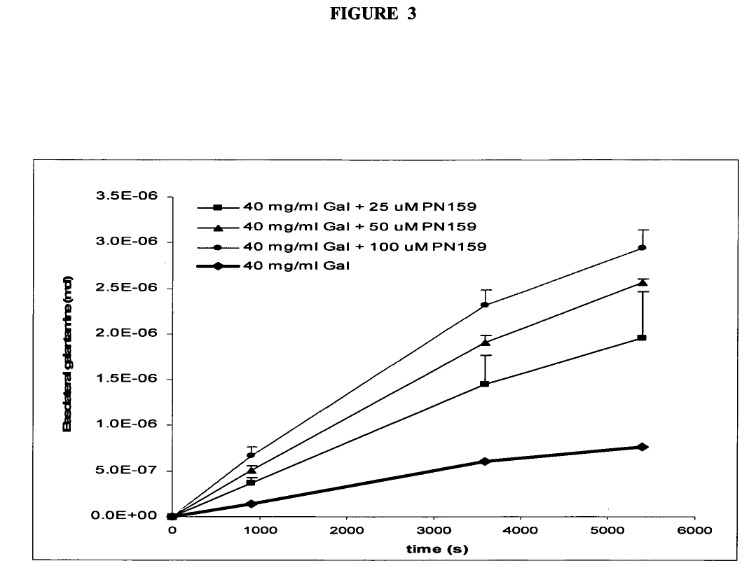

In the in vitro tissue model, the addition of PN159 resulted in a dramatic increase in drug permeation across the cell barrier. Specifically, there was a 2.5-3.5 fold increase in the Pₐₚₚ of 40 mg/ml galantamine. (FIG. 3)

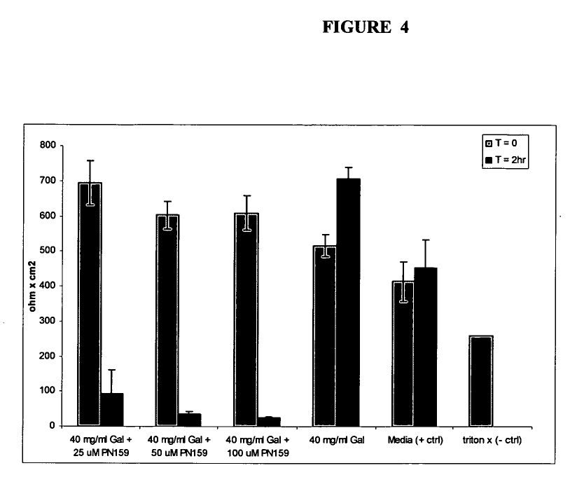

As shown in FIG. 4, PN159 successfully reduced TER at all three concentrations tested in the presence of galantamine. Media applied to the apical side did not reduce TER while triton X did reduce TER, as expected.

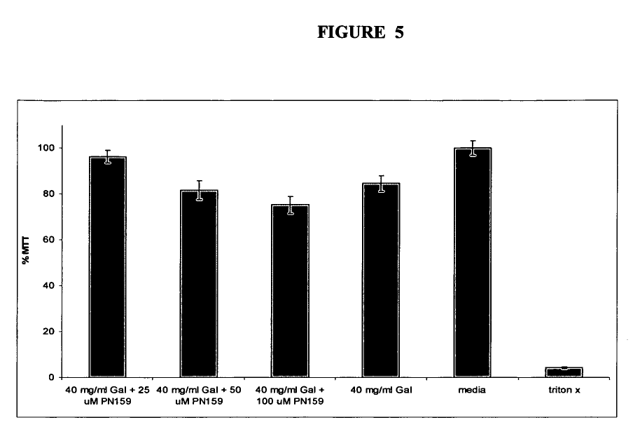

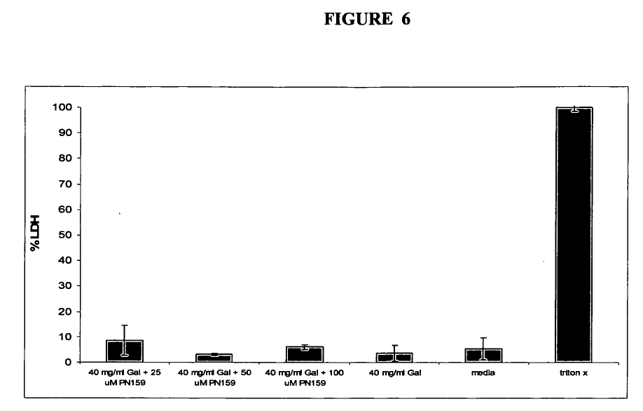

Cell viability remained high (>80%) in the presence of galantamine lactate and PN159 at all concentrations tested (FIG. 5). Media and triton X controls behaved as expected. Conversely, cyctotoxicity was low in the presence of PN159 and galantamine lactate, as measured by LDH. Again, media and triton X controls behaved as expected. (FIG. 6) Both of these assays indicate that PN159 is not unacceptably toxic to the epithelial membrane.

Summarizing the foregoing results, PN159 has been demonstrated herein to surprisingly increase epithelial permeation of galantamine as a model low molecular weight drug. The addition of PN159 to galantamine in solution significantly enhances galantamine permeation across epithelial monolayers. Evidence shows that PN159 temporarily reduces TER across the epithelial membrane without damaging the cells in the membrane, as measured by high cell viability and low cytotoxicity. PN159 therefore is an exemplary peptide for enhancing bioavailability of galantamine and other small molecule druges in vivo. It is further expected that PN159 will enhance permeation of galantamine and other small molecule drugs at higher concentrations as well.

### EXAMPLE 22

### Dosing of Intrnasal Vs. Oral Galantamine in Canine Subjects Results in decreased Tmax

The present example describes dosing of galantamine in dog subjects, which are accepted in the art as predictive model subjects for ACE inhibitor activity in humans, by intranasal versus oral delivery routes. The data provided here demonstrate that intranasal formulations of the invention result in increased speed of onset and/or higher Cmax, and/ro reduction in emetic response compared to oral dosing.

Study Design:

1 dog cross over (2 day wash out period)
3 dosings:
   0.8 mg/kg Galantamine lactate intranasal (80 mg/ml gal, 30 mg/ml Me-b-CD, 1.7 mg/ml DDPC, 2 mg/ml EDTA)
   0.8 mg/kg Reminyl® oral tablet
   0.8 mg/kg Reminyl® oral solution (0.8 mg/ml gal)
CSF and blood draws:
   0, 2.5, 5, 10, 15, 30 min, 1, 2, 4, hrs

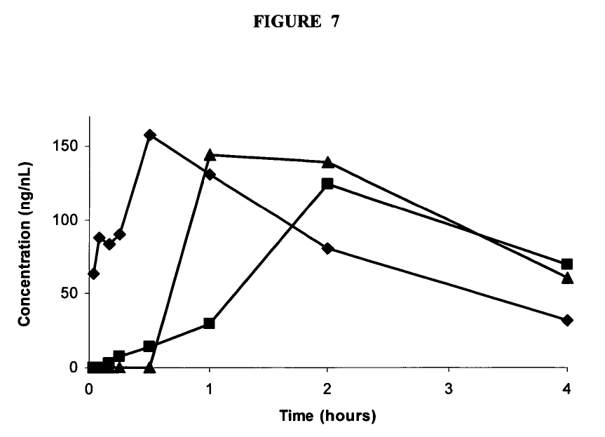

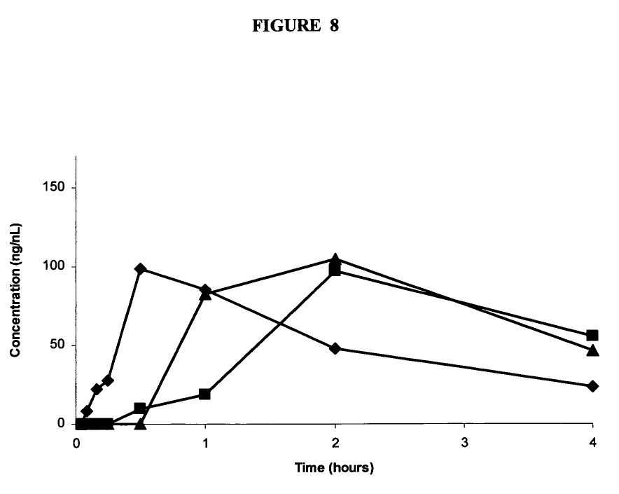

FIG. 7 illustrates the plasma levels of galantamine in a canine subject resulting from oral tablet, oral liquid and intranasal delivery. The data clearly show a decreased Tmax (about 30 min) vs. about 2-3 hours for the oral route. The same trend was observed in the CSF (FIG. 8). For therapeutic applications where speed of onset is important, e.g, neuropathic pain, a faster speed of onset is highly desirable.

### EXAMPLE 23

### Dosing of Intrnasal Vs. Oral Galantamine in Ferrets Results in decreased Tmax Increased Cmax, and Reduced Gastrointestinal (GI) Side Effects

The purpose of this study was to compare galantamine PK for oral vs intranasal administration, and also to examine the incidence of retching in ferret subjects that are accepted in the art as predictive of ACE inhibitor activity in humans.

Study Design

8 naïve ferrets/dosing group; Nasal versus Oral
Emetic Response Assay
The assay was based on a validated method. Ferrets were monitored and observed for episodes of retching and/or vomiting for 4 hours.
Two formulations dosed:
Emetic Response Assay
The assay was based on a validated method. Ferrets were monitored and observed for episodes of retching and/or vomiting for 4 hours.
Formulations tested:
   Intranasal: 200 mg/ml gal, 30 mg/ml Me-b-CD, 1.7 mg/ml DDPC, 2 mg/ml EDTA
   Oral: 20 mg/ml galantamine in Reminyl® oral solution

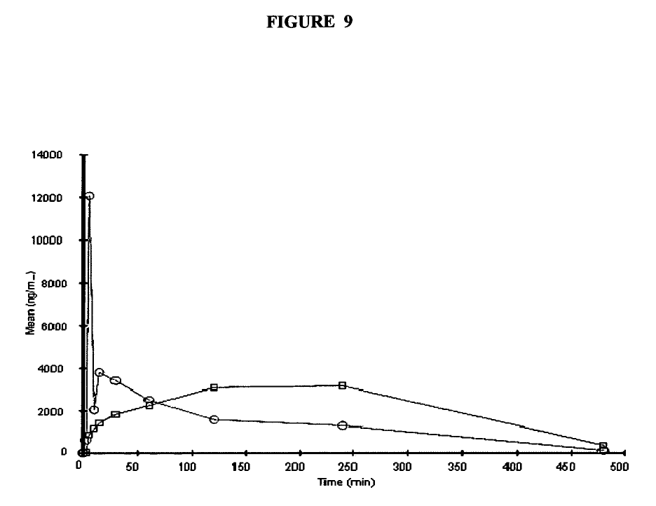

FIG. 9 illustrates PK (plasma) results in ferrets following intranasal vs. oral delivery. These data evince a decreased Tmax, and higher Cmax (about 4 fold) for intranasal as compared to oral delivery. The overall bioavailability of intranasal dosing compared to oral was about 70%.

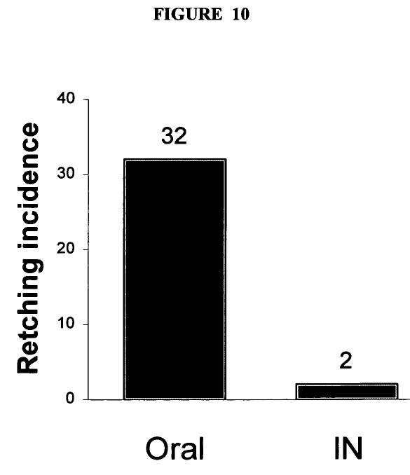

FIG. 10 shows the incidence of retching responses after administration of galantamine via different routes in ferrets. The data evince a dramatic reduction in emesis for intranasal vs. oral dosing.

Although the foregoing invention has been described in detail by way of example for purposes of clarity of understanding, it will be apparent to the artisan that certain changes and modifications may be practiced within the scope of the appended claims, which are presented by way of illustration not limitation. In this context, various publications and other references have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

1. A pharmaceutical composition for treatment or prevention of a central nervous system (CNS) disease or condition in a mammal amenable to treatment by therapeutic administration of an acetylcholinesterase (ACE) inhibitor, comprising:
a. a liquid, gel, or powder formulation for nasal administration comprising galantamine or a pharmaceutically acceptable salt thereof; and
b. at least one permeation-enhancing agent effective to enhance transmucosal drug uptake.

2. The pharmaceutical composition of claim 1, wherein said CNS disease or condition is Alzheimer's disease.

3. The pharmaceutical composition of claim 1, wherein said liquid or gel solution is an aqueous solution.

4. The pharmaceutical composition of claim 1, wherein said liquid solution is a solution in a liquid polyoxyethylene glycol.

5. The pharmaceutical composition of claim 1, wherein where said liquid solution is a solution in at least one liquid selected from the group consisting of dimethylsulfoxide, n-methylpyrrolidinone, transcutol, short chain diglyceride, and short chain mononoglyceride.

6. The pharmaceutical composition of claim 1, wherein said composition comprises a carboxylate salt form of galantamine.

7. The pharmaceutical composition of claim 6, wherein said carboxylate salt of galantamine is a lactate salt of galantamine.

8. The pharmaceutical composition of claim 6, wherein said said carboxylate salt of galantamine is a gluconate salt of galantamine.

9. The pharmaceutical composition of claim 6, wherein said carboxylate salt of galantamine is an acetate salt of galantamine.

10. The pharmaceutical composition of claim 6, wherein said carboxylate salt of galantamine is a citrate salt of galantamine.

11. The pharmaceutical composition of claim 6, wherein said carboxylate salt of galantamine is a glucarate salt of galantamine.

12. The pharmaceutical composition of claim 1, further comprising a co-therapeutic selected from the group consisting of a COX-2 inhibitor, huperzine (selegine) and 4,4′-diaminodiphenylsulfone.

13. The pharmaceutical composition of claim 1, wherein said pharmaceutical composition is substantially free of native neurobiomolecules selected from the group consisting of ganglioside, phosphatidylserine, brain-derived neurotropic factor, fibroblast growth factor, insulin, insulin-like growth factors, ciliary neurotropic factor, glia-derived nexin, cholinergic enhancing factors, phosphoethanolamine and thyroid hormone T3.

14. The pharmaceutical composition of claim 1, wherein said pharmaceutical composition is substantially free of G-1 ganglioside.

15. The pharmaceutical composition of claim 1, wherein said pharmaceutical composition is substantially free of native neurobiomolecules that stimulate nerve cell growth.

16. The pharmaceutical composition of claim 1, wherein said permeation-enhancing agent is selected from:
(a) an aggregation inhibitory agent;
(b) a charge modifying agent;
(c) a pH control or buffering agent;
(d) a redox control or buffering agent
(e) a degradative enzyme inhibitory agent;
(f) a mucolytic or mucus clearing agent;
(g) a ciliostatic agent;
(h) an absorption enhancement agent selected from (i) a surfactant, (ii) a bile salt, (iii) a phospholipid additive, mixed micelle, liposome, or carrier, (iv) an alcohol, (v) an enamine, (vi) an NO donor compound, (vii) a long-chain amphipathic molecule (viii) a small hydrophobic penetration enhancer; (ix) sodium or a salicylic acid derivative; (x) a glycerol ester of acetoacetic acid (xi) a cyclodextrin or β-cyclodextrin derivative, (xii) a medium-chain fatty acid, (xiii) a chelating agent, (xiv) an amino acid or salt thereof, (xv) an N-acetylamino acid or salt thereof, (xvi) an enzyme degradative to a selected membrane component, (xvii) an inhibitor of fatty acid synthesis, or (xviii) an inhibitor of cholesterol synthesis; or (xix) any combination of the membrane penetration enhancing agents recited in (i)-(xviii);
(i) a modulatory agent of epithelial junction physiology;
(j) a vasodilator agent;
(k) a stabilizing delivery vehicle, carrier, support or complex-forming species with which the acetylcholinesterase inhibitor is effectively combined, associated, contained, encapsulated or bound resulting in stabilization of the acetylcholinesterase inhibitor for enhanced mucosal delivery, wherein the formulation of said acetylcholinesterase inhibitor with said one or more delivery-enhancing agents provides for increased bioavailability of the acetylcholinesterase inhibitor in a central nervous system tissue or fluid of said subject;
(l) a humectant or membrane stabilizing agent; and
(m) a permeation-enhancing peptide agent.

17. The pharmaceutical composition of claim 1, comprising a plurality of said permeation-enhancing agents.

18. The pharmaceutical composition of claim 1, comprising a plurality of said absorption enhancing agents.

19. The pharmaceutical composition of claim 1, wherein said absorption enhancing agent is glycyrrhitic acid or a derivative thereof.

20. The pharmaceutical composition of claim 1, further comprising a chitosan or chitosan derivative.

21. The pharmaceutical formulation of claim 20, wherein said chitosan or chitosan derivative is poly-GuD.

22. The pharmaceutical composition of claim 1, wherein said pharmaceutical composition has a pH 3.0-6.0.

23. The pharmaceutical composition of claim 1, wherein said pharmaceutical composition has a pH 3.0-5.0.

24. The pharmaceutical composition of claim 1, wherein said pharmaceutical composition has a pH 3.0-4.0.

25. The pharmaceutical composition of claim 1, wherein said galantamine or pharmaceutical salt thereof is administered to said mammal in an effective dose of between about 0.1 mg and about 100 mg.

26. The pharmaceutical composition of claim 1, wherein said permeation-enhancing agent is selected from the group consisting of citric acid, sodium citrate, propylene glycol, glycerin, L-ascorbic acid, sodium metabisulfite, edetate disodium, benzalkonium chloride, sodium hydroxide and mixtures thereof.

27. The pharmaceutical composition of claim 1, wherein said galantamine or pharmaceutical salt thereof is a prodrug.

28. The pharmaceutical composition of claim 1, further comprising a membrane stabilizing agent to reduce nasal irritation.

29. The pharmaceutical composition of claim 1, wherein said membrane stabilizing agent to reduce nasal irritation is vitamin E or a derivative of vitamin E.

30. The pharmaceutical composition of claim 1, wherein said pharmaceutical composition following intranasal adminstration to said mammal yields a peak concentration of said galantamine or pharmaceutical salt thereof in a central nervous system tissue or fluid of said mammal that is at least equal to a therapeutic plasma concentration of said galantamine or pharmaceutical salt thereof in a blood plasma of said mammal.

31. The pharmaceutical composition of claim 1, wherein said permeation-enhancing agent is a permeabilizing peptide.

32. The pharmaceutical composition of claim 31, wherein said permeabilizing peptide reversibly enhances mucosal epithelial paracellular transport of said galantamine or pharmaceutically acceptable salt thereof.

33. The pharmaceutical composition of claim 31, wherein said permeabilizing peptide is selected from the following group of peptides, or comprises an active fragment, conjugate, or complex thereof:
(SEQ ID NO: 1)
RKKRRQRRRPPQCAAVALLPAVLLALLAP;
(SEQ ID NO: 2)
RQIKIWFQNRRMKWKK;
(SEQ ID NO: 3)
GWTLNSAGYLLGKINLKALAALAKKIL;
(SEQ ID NO: 4)
KLALKLALKALKAALKLA;
(SEQ ID NO: 5)
KLWSAWPSLWSSLWK;
(SEQ ID NO: 6)
AAVALLPAVLLALLAPRKKRRQRRRPPQ;
(SEQ ID NO: 7)
LLETLLKPFQCRICMRNFSTRQARRNHRRRHRR;
(SEQ ID NO: 8)
RRRQRRKRGGDIMGEWGNEIFGAIAGFLG;
(SEQ ID NO: 9)
KETWWETWWTEWSQPGRKKRRQRRRPPQ;
(SEQ ID NO: 10)
GLGSLLKKAGKKLKQPKSKRKV;
and
(SEQ ID NO: 11)
KGSKKAVTKAQKKDGKKRKRSRKESYSVYVYKVLKQ.

34. The pharmaceutical composition of claim 1, wherein said nasal administration involves delivery of said composition to one or both nasal mucosal surfaces of said mammal.

35. A method for treating or preventing a disease or condition in a mammal in need of treatment by therapeutic administration of galantamine or a pharmaceutically acceptable salt thereof, comprising the step of administering intranasally to said mammal a pharmaceutical composition of claim 1.

36. The method of claim 35, wherein said pharmaceutical composition is administered as a single solution in a multidose nasal dispenser.

37. The method of claim 35, wherein said disease or condition amenable to treatment by therapeutic administration of said galantamine or pharmaceutically acceptable salt thereof is Alzheimer's disease.

38. The method of claim 35, wherein said disease or condition amenable to treatment by therapeutic administration of said galantamine or pharmaceutically acceptable salt thereof is Parkinson's-like dementia.

39. The method of claim 35, wherein said disease or condition amenable to treatment by therapeutic administration of said galantamine or pharmaceutically acceptable salt thereof is Huntington's-type dementia.

40. The method of claim 35, wherein said disease or condition amenable to treatment by therapeutic administration of said galantamine or pharmaceutically acceptable salt thereof is Pick's-type dementia.

41. The method of claim 35, wherein said disease or condition amenable to treatment by therapeutic administration of said galantamine or pharmaceutically acceptable salt thereof is AIDS related dementia or delirium.

42. The method of claim 35, wherein said disease or condition amenable to treatment by therapeutic administration of said galantamine or pharmaceutically acceptable salt thereof is dementia secondary to vascular disorder.

43. The method of claim 35, wherein said disease or condition amenable to treatment by therapeutic administration of said galantamine or pharmaceutically acceptable salt thereof is moderate cognitive impairment.

44. The method of claim 35, wherein said disease or condition amenable to treatment by therapeutic administration of said galantamine or pharmaceutically acceptable salt thereof is Crutzfeld-Jacobsen type dementia.

45. The method of claim 35, wherein said disease or condition amenable to treatment by therapeutic administration of said galantamine or pharmaceutically acceptable salt thereof is a learning disorder.

46. The method of claim 35, wherein said disease or condition amenable to treatment by therapeutic administration of said galantamine or pharmaceutically acceptable salt thereof is nicotine withdrawal syndrome.

47. The method of claim 35, wherein said administration involves delivery of said pharmaceutical composition to a nasal mucosal surface of said mammal.

48. The method of claim 35, wherein said composition comprises a carboxylate salt form of galantamine.

49. The method of claim 48, wherein said carboxylate salt of galantamine is a lactate or gluconate salt of galantamine.

50. The method of claim 35, wherein said galantamine or pharmaceutically acceptable salt thereof is administered to said mammal in an effective dose of between about 0.1 mg and 100 mg.

51. The method of claim 35, wherein said permeation-enhancing agent for transmucosal drug uptake is selected from:
(a) an aggregation inhibitory agent;
(b) a charge modifying agent;
(c) a pH control or buffering agent;
(d) a redox control or buffering agent
(e) a degradative enzyme inhibitory agent;
(f) a mucolytic or mucus clearing agent;
(g) a ciliostatic agent;
(h) an absorption enhancement agent selected from (i) a surfactant, (ii) a bile salt, (iii) a phospholipid additive, mixed micelle, liposome, or carrier, (iv) an alcohol, (v) an enamine, (vi) an NO donor compound, (vii) a long-chain amphipathic molecule (viii) a small hydrophobic penetration enhancer; (ix) sodium or a salicylic acid derivative; (x) a glycerol ester of acetoacetic acid (xi) a cyclodextrin or β-cyclodextrin derivative, (xii) a medium-chain fatty acid, (xiii) a chelating agent, (xiv) an amino acid or salt thereof, (xv) an N-acetylamino acid or salt thereof, (xvi) an enzyme degradative to a selected membrane component, (xvii) an inhibitor of fatty acid synthesis, or (xviii) an inhibitor of cholesterol synthesis; or (xix) any combination of the membrane penetration enhancing agents recited in (i)-(xviii); (i) a modulatory agent of epithelial junction physiology;
(j) a vasodilator agent;
(k) a stabilizing delivery vehicle, carrier, support or complex-forming species with which the acetylcholinesterase inhibitor is effectively combined, associated, contained, encapsulated or bound resulting in stabilization of the acetylcholinesterase inhibitor for enhanced mucosal delivery, wherein the formulation of said acetylcholinesterase inhibitor with said one or more delivery-enhancing agents provides for increased bioavailability of the acetylcholinesterase inhibitor in a central nervous system tissue or fluid of said subject;
(l) a humectant or membrane stabilizing agent; and
(m) a permeation-enhancing peptide agent.

52. The method of claim 35, wherein said permeation-enhancing agent is selected from the group consisting of citric acid, sodium citrate, propylene glycol, glycerin, L-ascorbic acid, sodium metabisulfite, edetate disodium, benzalkonium chloride, sodium hydroxide and mixtures thereof.

53. The method of claim 35, wherein said permeation-enhancing agent is a permeabilizing peptide.

54. The method of claim 35, wherein said permeabilizing peptide reversibly enhances mucosal epithelial paracellular transport of said galantamine or pharmaceutically acceptable salt thereof.

55. The method of claim 35, wherein said permeabilizing peptide is selected from the following group of peptides, or comprises an active fragment, conjugate, or complex thereof:
(SEQ ID NO: 1)
RKKRRQRRRPPQCAAVALLPAVLLALLAP;
(SEQ ID NO: 2)
RQIKIWFQNRRMKWKK;
(SEQ ID NO: 3)
GWTLNSAGYLLGK1NLKALAALAKKIL;
(SEQ ID NO: 4)
KLALKLALKALKAALKLA;
(SEQ ID NO: 5)
KLWSAWPSLWSSLWKP;
(SEQ ID NO: 6)
AAVALLPAVLLALLAPRKKRRQRRRPPQ;
(SEQ ID NO: 7)
LLETLLKPFQCRICMRNFSTRQARRNHRRRHRR;
(SEQ ID NO: 8)
RRRQRRKRGGDIMGEWGNEIFGAIAGFLG;
(SEQ ID NO: 9)
KETWWETWWTEWSQPGRKKRRQRRRPPQ;
(SEQ ID NO: 10)
GLGSLLKKAGKKLKQPKSKRKV;
and
(SEQ ID NO: 11)
KGSKKAVTKAQKKDGKKRKRSRKESYSVYVYKVLKQ.

56. The method of claim 35, wherein said pharmaceutical composition following intranasal adminstration to said mammal yields a peak concentration of said galantamine or pharmaceutically acceptable salt thereof in a central nervous system tissue or fluid of said mammal that is at least 15% of the peak concentration of said galantamine or pharmaceutically acceptable salt thereof in a blood plasma of said mammal.

57. The method of claim 35, wherein said pharmaceutical composition following intranasal adminstration to a mammal yields a peak concentration of said galantamine or pharmaceutically acceptable salt thereof in a central nervous system tissue or fluid of said mammal that is at least 30% of the peak concentration of said galantamine or pharmaceutically acceptable salt thereof in a blood plasma of said mammal.

58. The method of claim 35, wherein said pharmaceutical composition following intranasal adminstration to said mammal yields a peak concentration of said galantamine or pharmaceutically acceptable salt thereof in a central nervous system tissue or fluid of said mammal that is at least 40% of the peak concentration of said galantamine or pharmaceutically acceptable salt thereof in a blood plasma of said mammal.

59. The method of claim 35, wherein said pharmaceutical composition following mucosal adminstration to said mammal yields a peak concentration of said galantamine or pharmaceutically acceptable salt thereof in a central nervous system tissue or fluid of said subject that is greater than a therapeutic concentration of said galantamine or pharmaceutically acceptable salt thereof in the plasma of said subject.

60. The method of claim 35, wherein said pharmaceutical composition following intranasal adminstration to said mammal yields a reduction of an emetic response in said mammal in comparison to an emetic response observed in mammals following oral administration of an equivalent amount of said galantamine or pharmaceutically acceptable salt thereof.

61. An article of manufacture, comprising:
a. a means for administering a nasal dose; and
b. the composition of claim 1.

62. The article of manufacture of claim 61, wherein said means for administering a nasal dose is a nasal dispenser, tampon, sponge, insufflator, nebulizer or pump.

63. An article of manufacture comprising the pharmaceutical composition of claim 1 in a package suitable for sale and distribution.

64. A galantamine carboxylate salt.

65. The galantamine carboxylate salt of claim 64, having a carboxylate anion associated with a galantamine cation in solution.

66. The galantamine carboxylate salt of claim 64, wherein the carboxylate anion contains one or more hydroxyl groups.

67. A galantamine carboxylate salt selected from the group consisting of galantamine gluconate, galantamine lactate, galantamine citrate and galantamine glucarate, galantamine benzoate, galantamine acetate, galantamine salicylate, galantamine tartrate, galantamine mesylate, galantamine tosylate, galantamine maleate, galantamine fumarate, and galantamine stearate.

68. A method for producing a galantamine carboxylate salt comprising:
forming a solution of a carboxylate salt forming carboxylate anions in solution;
applying the solution to an anion exchange resin under conditions wherein the carboxylate anions bind to the anion exchange resin;
forming a solution of galantamine hydrobromide under conditions where bromide ions are formed in solution; and
applying the galantamine hydrobromide solution to the anion exchange resin under conditions wherein the carboxylate anions are displaced and the bromide anions bind to the anion exchange resin resulting in the formation of a galantamine carboxylate salt or complex.

69. The method of claim 68, wherein the process is conducted in a batch process.

70. The method of claim 69, wherein the batch process is a slurry of anion exchange resin.

71. The method of claim 69, wherein the batch process uses an anion exchange resin packed into a column.

72. The method of claim 71, wherein the process is conducted as a continuous process using an anion exchange resin packed into a column.

73. The method of claim 68, wherein the carboxylate salt is selected from the group consisting of a gluconate salt, an acetate salt, a citrate salt and a glucarate salt.

74. The method of claim 73, wherein the process is conducted in a batch process.

75. The method of claim 74, wherein the batch process is a slurry of the anion exchange resin.

76. The method of claim 74, wherein the batch process uses an anion exchange resin packed into a column.

77. The method of claim 76, wherein the process is conducted as a continuous process using an anion exchange resin packed into a column.

78. A method for inhibiting acetylcholinesterase in a mammal comprising administering to said individual a galantamine carboxylate salt.

79. The method of claim 78, wherein the mammal is a human.

80. The method of claim 78, wherein the galantamine carboxylate salt is selected from the group consisting of galantamine gluconate, galantamine lactate, galantamine citrate and galantamine glucarate, galantamine benzoate, galantamine acetate, galantamine salicylate, galantamine tartrate, galantamine mesylate, galantamine tosylate, galantamine maleate, galantamine fumarate, and galantamine stearate.

81. The method of claim 80, wherein the mammal is a human.

82. The method claim 78, wherein galantamine carboxylate salt has carboxylate anion associated with a galantamine cation in solution.

83. The method of claim 82, wherein the mammal is a human.

84. The method of claim 82, wherein the carboxylate anion contains one or more hydroxyl groups.

85. The method of claim 84, wherein the mammal is a human.
